SEQUENCE LISTING (25 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - DNA, 13 bp, synthetic construct
  source 1..13: mol_type = other DNA
gccgccaccatgg

SEQ ID NO: 3 - protein, 25 aa, synthetic construct
MGVLLTQRTLLSLVLALLFPSMASM

SEQ ID NO: 4 - protein, 19 aa, synthetic construct
MGWSCIILFLVATATGVHS

SEQ ID NO: 5 - protein, 21 aa, synthetic construct
MWWRLWWLLLLLLLLWPMVWA

SEQ ID NO: 6 - protein, 22 aa, synthetic construct
MDMRVPAQLLGLLLLWLRGARC

SEQ ID NO: 7 - protein, 16 aa, synthetic construct
MPLLLLLPLLWAGALA

SEQ ID NO: 8 - protein, 23 aa, synthetic construct
MDAMKRGLCCVLLLCGAVFVSPS

SEQ ID NO: 9 - protein, 18 aa, synthetic construct
MAFLWLLSCWALLGTTFG

SEQ ID NO: 10 - protein, 15 aa, synthetic construct
MNLLLILTFVAAAVA

SEQ ID NO: 11 - protein, 20 aa, synthetic construct
MYRMQLLSCIALSLALVTNS

SEQ ID NO: 12 - protein, 16 aa, synthetic construct
MKWVTFISLLFSSAYS

SEQ ID NO: 13 - protein, 20 aa, synthetic construct
MASHRLLLLCLAGLVFVSEA

SEQ ID NO: 14 - protein, 27 aa, synthetic construct
MARRSSFQSCQIISLFTFAVGVNICLG

SEQ ID NO: 15 - protein, 24 aa, synthetic construct
MALWMRLLPLLALLALWGPDPAAA

SEQ ID NO: 16 - protein, 24 aa, synthetic construct
MPSSVSWGILLLAGLCCLVPVSLA

SEQ ID NO: 17 - DNA, 20 bp, synthetic construct
  source 1..20: mol_type = other DNA
tgcatctggatctggctggc

SEQ ID NO: 18 - DNA, 20 bp, synthetic construct
  source 1..20: mol_type = other DNA
tcatgcatctggatctggct

SEQ ID NO: 19 - DNA, 20 bp, synthetic construct
  source 1..20: mol_type = other DNA
atcatgcatctggatctggc

SEQ ID NO: 20 - DNA, 20 bp, synthetic construct
  source 1..20: mol_type = other DNA
tgccatcatgcatctggatc

SEQ ID NO: 21 - DNA, 20 bp, synthetic construct
  source 1..20: mol_type = other DNA
agccagatccagatgcatga

SEQ ID NO: 22 - protein, 11 aa, synthetic construct
KLWVLPKGGGC

SEQ ID NO: 23 - protein, 11 aa, synthetic construct
CGGGLSLITRL

SEQ ID NO: 24 - protein, 15 aa, synthetic construct
CRHSQMTVTSRLGGG

SEQ ID NO: 25 - protein, 20 aa, synthetic construct
CGGGANTPCGPYTHDCPVKR

(19) United States
(12) Patent Application Publication
(10) Pub. No.: US 2025/0312482 A1
(43) Pub. Date: Oct. 9, 2025

(54) LIPID NANOPARTICLES FOR THE TREATMENT OF VASCULAR DISEASES

(71) Applicants: The Brigham and Women's Hospital, Inc., Boston, MA (US); The General Hospital Corporation, Boston, MA (US)

(72) Inventors: Natalie Artzi, Brookline, MA (US); Rajeev Malhotra, Boston, MA (US); Mark Evan Lindsay, Winchester, MA (US); Eliz Amar-Lewis, Newton, MA (US); Patricia L. Musolino, Boston, MA (US); Christian Lacks Lino Cardenas, Boston, MA (US)

(21) Appl. No.: 19/171,160

(22) Filed: Apr. 4, 2025

Related U.S. Application Data
(60) Provisional application No. 63/574,782, filed on Apr. 4, 2024.
(60) Provisional application No. 63/574,833, filed on Apr. 4, 2024.
(60) Provisional application No. 63/719,028, filed on Nov. 11, 2024.
(60) Provisional application No. 63/719,040, filed on Nov. 11, 2024.

(51) Int. Cl. A61K 48/00; A61K 38/46; A61K 47/54; A61K 47/60; A61K 47/64; A61K 47/69

(52) U.S. Cl. CPC A61K 48/0033; A61K 38/465; A61K 47/543; A61K 47/60; A61K 47/64; A61K 47/6925

(57) ABSTRACT

Described herein are lipid nanoparticle (LNP) formulations with demonstrated tropism towards smooth muscle cells. Also described herein are LNPs conjugated with peptides that can target tissue or cell surface receptors. The formulations of the disclosure include amounts of DOTAP, an ionizable lipid, amounts of a neutral lipid; amounts of cholesterol; and amounts of one or more PEG-lipids with preferential tropism towards vascular smooth muscle cells (vSMCs). Also described herein are peptides that target receptors highly expressed on the surface of vSMCs (IL-6R, CD63 and GAL-3) or that target proteins in the extracellular matrix adjacent to vSMCs (Col-IV) increasing the uptake into these cells.

### CLAIM OF PRIORITY

This application claims the benefit of U.S. Provisional Application Ser. No. 63/574,782, filed on Apr. 4, 2024, 63/719,028, filed on Nov. 11, 2024, 63/574,833, filed on Apr. 4, 2024, and 63/719,040, filed on Nov. 11, 2024. The entire contents of the foregoing are incorporated herein by reference.

### SEQUENCE LISTING

This application contains a Sequence Listing that has been submitted electronically as an XML file named “29618-0486001_SL_ST26.XML.” The XML file, created on Apr. 8, 2025, is 23,870 bytes in size. The material in the XML file is hereby incorporated by reference in its entirety.

### TECHNICAL FIELD

Lipid nanoparticles (LNPs) are biodegradable and biocompatible nanostructures, that are in principle capable of encapsulating nucleic acids with high efficiency and delivery into cells. However, the delivery of large nucleic acids such as plasmids has been more challenging and in particular, cell-specific delivery remains a challenge.

### BACKGROUND

The current state of the art for therapeutic nucleic acid delivery is lipid nanoparticles (LNP), which are composed of cholesterol, a helper lipid, a PEGylated lipid and an ionizable amine-containing lipid. The liver, however, is the primary organ of LNP accumulation following intravenous administration and is also observed to varying degrees following intramuscular and subcutaneous routes. What this generally means is that higher concentrations of a therapeutic are typically required for effective administration of a treatment to a non-liver target cell. Formulating therapeutic nucleic acids into nanoparticles is of utmost importance to prevent degradation by nucleases upon administration and to enhance cellular uptake of these negatively charged entities.

The cholesterol and helper lipids are important for the integrity of the LNP, while the PEGylated lipid provides colloidal stability as well as stealth properties to limit accumulation in the reticuloendothelial system (RES). The ionizable amine-containing lipid is responsible for the complexation of nucleic acid. Importantly, this ionizable lipid is only protonated at non-physiological pH, pKa 6-7, which means the lipid is not charged in the circulation, which is important as cationic nanoparticles are notoriously toxic. Upon cell uptake and lysosomal localization, the ionizable lipid is again charged at the low lysosomal pH, which, together with the unique conical features of the component lipids, assists in lysosomal escape and mRNA expression or siRNA gene silencing. However, the art provides limited understanding of how such widely used formulations are, or can be, distributed within the body of a subject upon administration.

### SUMMARY

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

In some aspects, the disclosure provides a particle comprising: from 0.1% to 80% of a molecule of formula I

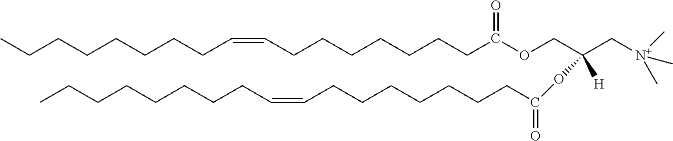 [SMILES: CCCCCCCC/C=C\CCCCCCCC(=O)OC[C@H](C[N+](C)(C)C)OC(=O)CCCCCCC/C=C\CCCCCCCC]

(DOTAP); and a nucleic acid therapeutic cargo encoding a gene for rescuing or correcting gene expression in a smooth muscle cell. In some aspects, the particle further comprises further an amount of an ionizable lipid; an amount of neutral lipid; an amount of cholesterol; an amount of one or more PEG-lipids; whereby amounts of the ionizable lipid, the neutral lipid, the cholesterol, and the one or more PEG-lipids are at a molar ratio of 10:2.1:7.6:1.5 of the remaining weight of the particle. In some aspects, the particle further comprises from 7% up to 13% of an ionizable lipid; from 1% up to 3% of a neutral lipid; from 6% up to 8% of cholesterol; from 0.1% up to 2% of one or more PEG-lipids of a percentage of total lipids in the particle.

In some instances, a PEG-lipid of the one or more PEG-lipids is a maleimide-terminally modified PEG lipid. In some instances, the particle further comprises a peptide conjugated to the particle via the maleimide-terminally modified PEG lipid. The peptide can be a peptide targeting collagen IV (Col-IV) peptide or a functional fragment thereof, a peptide targeting IL-6R or a functional fragment thereof, a peptide targeting CD63 or a functional fragment thereof, or a peptide targeting GAL-3 or a functional fragment thereof. In some instances, the particle further comprises one or more peptides targeting collagen IV (Col-IV), IL-6R, CD63, GAL-3, or any combination thereof. The peptide can be selected from the group consisting of SEQ ID NO: 22, SEQ ID NO: 23, SEQ ID NO: 24, and SEQ ID NO: 25. In some instances, the neutral lipid is a phosphatidylcholine lipid or a phosphatidylethanolamine lipid. In some instances, phosphatidylcholine lipid or the phosphatidylethanolamine lipid is selected from the group consisting of DSPC, DPPC, and POPC. In some instances, the ionizable lipid is selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DM A, DLin-K-C4-DMA, DLen-C2K-DMA, 7-DLen-C2K-DMA, or DLin-MP-DMA. In some instances, the therapeutic cargo is an mRNA molecule encoding a gene, optionally a gene for rescuing gene expression in the smooth muscle cell. In some instances, the therapeutic cargo is a plasmid encoding a gene, optionally a gene for rescuing gene expression in the smooth muscle cell; e.g., a nucleic acid molecule encoding an ectonucleotide pyrophosphatase/phosphodiesterase 1 (ENPP1) therapeutic cargo, a nucleic acid molecule encoding an ATP Binding Cassette Subfamily C Member 6 (ABCC6) therapeutic cargo, or a nucleic acid molecule encoding an Actin Alpha 2 (ACTA2) gene. In some instances, the ENPP1 therapeutic cargo encodes a transmembrane ENPP1 molecule. In some instances, the transmembrane ENPP1 molecule is SEQ ID NO: 1. In some instances, the ENPP1 therapeutic cargo encodes a soluble ENPP1 molecule, e.g., amino acids 103-925, and optionally amino acids 97-925 of SEQ ID NO: 1.

In some aspects, the disclosure provides a particle comprising: from 0.1% to 80% of a molecule of formula I

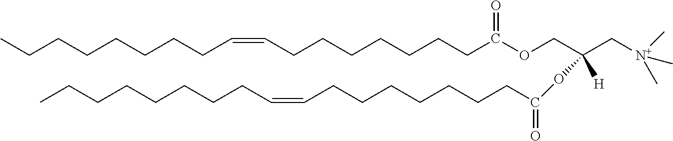 [SMILES: CCCCCCCC/C=C\CCCCCCCC(=O)OC[C@H](C[N+](C)(C)C)OC(=O)CCCCCCC/C=C\CCCCCCCC]

(DOTAP); and a peptide conjugated to a linker in the particle.

In some embodiments, the disclosure provides a particle comprising: a) an amount of an ionizable lipid; an amount of neutral lipid; an amount of cholesterol; and an amount of one or more PEG-lipids; an amount of a DOTAP molecule; and b) a peptide conjugated to a linker in the particle.

In some aspects, the linker is a maleimide group at a PEG lipid of the one or more PEG-lipids in the particle. In some embodiments, the linker is a maleimide-terminally modified PEG lipid. In some embodiments, the one or more PEG-lipids comprise 1,2-dimyristoyl-rac-glycero-3-methoxypolyethylene glycol (DMG-PEG) and 1,2-distearoyl-sn-glycero-3-phosphoethanolamine-N-[maleimide(polyethylene glycol](DSPE-PEG-maleimide). In some aspects, the peptide is a peptide targeting collagen IV (Col-IV) or a functional fragment thereof. The peptide can be a peptide targeting collagen IV (Col-IV) peptide or a functional fragment thereof, a peptide targeting IL-6R or a functional fragment thereof, a peptide targeting CD63 or a functional fragment thereof, or a peptide targeting GAL-3 or a functional fragment thereof. In some instances, the particle further comprises two or more, three or more, or all four peptides targeting collagen IV (Col-IV), IL-6R, CD63, and GAL-3, or any combination thereof.

In some aspects, the particle further comprises an amount of an ionizable lipid; an amount of neutral lipid; an amount of cholesterol; and an amount of one or more PEG-lipids. Such amounts can be, e.g., at a molar ratio of 10:2.1:7.6:1.5. In some embodiments, the particle comprises amounts of the ionizable lipid, the neutral lipid, the cholesterol, the one or more PEG-lipids, and DOTAP at a molar ratio of 10:2.1:7.6:1.5:78.8.

In some instances, the neutral lipid is a phosphatidylcholine lipid or a phosphatidylethanolamine lipid. In some instances, the phosphatidylcholine lipid or the phosphatidylethanolamine lipid is selected from the group consisting of DOPE, DOPC, DSPC, DPPC, POPC, and SOPC. In some instances the ionizable lipid is selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DM A, DLin-K-C4-DMA, DLen-C2K-DMA, 7-DLen-C2K-DMA, or DLin-MP-DMA.

In some instances, the particle encapsulates a nucleic acid therapeutic cargo. In some instances, the therapeutic cargo is an mRNA molecule encoding a gene, optionally a gene for rescuing gene expression in the smooth muscle cell. In some instances, the therapeutic cargo is a plasmid encoding a gene, optionally a gene encoding a gene for rescuing gene expression in the smooth muscle cell. In some instances, the therapeutic cargo is a plasmid encoding a gene for rescuing gene expression in the smooth muscle cell; e.g., a nucleic acid molecule encoding an ectonucleotide pyrophosphatase/phosphodiesterase 1 (ENPP1) therapeutic cargo, a nucleic acid molecule encoding an ATP Binding Cassette Subfamily C Member 6 (ABCC6) therapeutic cargo, or a nucleic acid molecule encoding an Actin Alpha 2 (ACTA2) gene, or a nucleic acid encoding a gene editing protein as described herein.

In some embodiments, the therapeutic cargo comprises a nucleic acid molecule encoding an ectonucleotide pyrophosphatase/phosphodiesterase 1 (ENPP1) therapeutic cargo, a nucleic acid molecule encoding an ATP Binding Cassette Subfamily C Member 6 (ABCC6) therapeutic cargo, a nucleic acid molecule encoding an Actin Alpha 2 (ACTA2) gene, or a nucleic acid encoding a genome editing protein.

In some instances, the ENPP1 therapeutic cargo encodes a transmembrane ENPP1 molecule. In some embodiments, the transmembrane ENPP1 molecule is SEQ ID NO: 1. In some embodiments, the ENPP1 therapeutic cargo encodes a soluble ENPP1 molecule. In some embodiments, the soluble ENPP1 molecule comprises amino acids 103-925, and optionally amino acids 97-925 of SEQ ID NO: 1 In some instances, the therapeutic cargo encodes (i) a CRISPR/Cas base editor or an intein-split construct thereof, and an Arg179His or an Arg179Cys mutant-allele specific guide RNA directing the base editor to the mutation, or
(ii) a CRISPR/Cas genome editor or an intein-split construct thereof, and an Arg179His or an Arg179Cys mutant-allele specific guide RNA directing the genome editor to the mutation. In some embodiments, the base editor or intein-split construct thereof, genome editor or intein-split construct thereof, and/or guide RNA target ACTA2 and are listed in Table A and/or Table B of WO2024073715 (comprising SEQ ID NOs:55-79 of WO2024073715), optionally comprising SEQ ID NO:60 of WO2024073715. In some embodiments, the Arg179His or the Arg179Cys mutant-allele specific CRISPR/Cas base editor is an adenine base editor or an intein-split construct thereof comprising the wild-type SpCas9, or D1135L/S1136W/G1218K/E1219Q/R1335Q/T1337R (SpG), A61R/L1111R/D1135L/S1136W/G1218K/E1219Q/N1317R/A1322R/R1333P/R1335 Q/T1337R (SpRY), D10T(optional)/I322V/S409/E427G/R654L/R753G/R1114G/D1135N/V1139A/D1180G/E1219 V/Q1221H/A1320V/R1333K (SpCas9-NRRH), D10T(optional)/I322V/S409I/E427G/R654L/R753G/R1114G/D1135N/E1219V/D1332N/R1335 Q/T1337N/S1338T/H1349R (SpCas9-NRCH), D1135M/S1136Q/G1218K/E1219S/R1335E/T1337R (MQSKER), D1135V/G1218R/R1335Q/T1337R (VRQR), or S55R/D1135V/G1218R/R1335Q/T1337R (VRQR(S55R)) variants of Streptococcus pyogenes Cas9 protein (SpCas9). In some embodiments, the spacer sequence of the guide RNA targets the sequence TGCATCTGGATCTGGCTGGC (SEQ ID NO:17) (HES1208-A4 gRNA) with a CGA PAM and the target adenine in position 4 of the spacer, e.g., optionally with ABE8e-SpCas9-VRQR, ABE8e-SpCas9-VRQR(S55R), ABE8e-SpG, ABE8e-SpRY, or ABE8e-SpCas9-NRRH (Table 1) or an intein-split construct thereof, or the sequence TCATGCATCTGGATCTGGCT (SEQ ID NO: 18, HES1210-A7 gRNA) with a GGC PAM and the target adenine in position A7 of the spacer, optionally with ABE8e-SpG, ABE8e-SpRY, ABE8e-SpCas9-NRCH, ABE8e-MQSKER (Table 1) or an intein-split construct thereof, or the sequence ATCATGCATCTGGATCTGGC (SEQ ID NO:19, HES1212-A8 gRNA) with a TGG PAM and the target adenine in position A8 of the spacer, optionally with ABE8e-SpCas9, ABE8e-SpG, ABE8e-SpRY, or ABE8e-SpCas9-NRRH (Table 1) or an intein-split construct thereof.

In some embodiments, the genome editor is a wild-type SpCas9 nuclease or an intein-split construct thereof, and in some embodiments, the spacer sequence of the guide RNA targets the sequence TGCCATCATGCATCTGGATC (HES1235, SEQ ID NO:20) or AGCCAGATCCAGATGCATGA (HES51236, SEQ ID NO:21). See WO/2024/073715.

In some aspects, the disclosure provides a therapeutic formulation comprising from 0.1% to 80% of a molecule of formula I (DOTAP) or a salt thereof, an amount of an ionizable lipid; an amount of neutral lipid; an amount of cholesterol; an amount of a PEG-lipid; comprising amounts of the ionizable lipid, the neutral lipid, the cholesterol, and the one or more PEG-lipids at a molar ratio of 10:2.1:7.6:1.5 and a nucleic acid construct encoding an ectonucleotide pyrophosphatase/phosphodiesterase 1 (ENPP1) therapeutic cargo.

In some aspects, the disclosure provides a therapeutic formulation comprising from about 10% of an MC3 ionizable lipid; about 2% of a DOPE neutral lipid; about 7% of cholesterol; about 1.5% of the one or more PEG-lipids; and a nucleic acid construct encoding an ectonucleotide pyrophosphatase/phosphodiesterase 1 (ENPP1) therapeutic cargo. In some embodiments, the ENPP1 therapeutic cargo is a transmembrane ENPP1 molecule. In some embodiments, the transmembrane ENPP1 molecule is SEQ ID NO: 1.

In some aspects, the disclosure provides a therapeutic formulation comprising: a) about 80% of DOTAP; b) about 10% of an MC3 ionizable lipid; c) about 2% of a DOPE neutral lipid; d) about 7% of cholesterol; e) about 1.5% of one or more PEG-lipids; and f) a nucleic acid construct encoding an ectonucleotide pyrophosphatase/phosphodiesterase 1 (ENPP1) therapeutic cargo. In some embodiments, the ENPP1 therapeutic cargo is a transmembrane ENPP1 molecule. In some embodiments, the transmembrane ENPP1 molecule is SEQ ID NO: 1.

In some aspects, the disclosure provides a method of delivering a nucleic acid therapeutic cargo to a smooth muscle cell, the method comprising administering to or contacting the smooth muscle cell with any of the particles or any of the therapeutic formulations described herein.

In some aspects, the disclosure provides a particle comprising: from 0.1% to 80% of a molecule of formula I

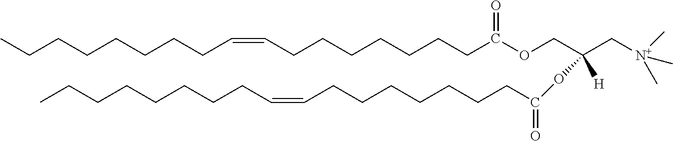 [SMILES: CCCCCCCC/C=C\CCCCCCCC(=O)OC[C@H](C[N+](C)(C)C)OC(=O)CCCCCCC/C=C\CCCCCCCC]

(DOTAP); and a nucleic acid therapeutic cargo encoding a gene for rescuing gene expression in a smooth muscle cell.

In some aspects, the disclosure provides a particle comprising: from 78.8% to 80% of a molecule of formula I

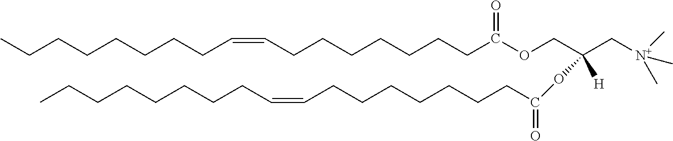 [SMILES: CCCCCCCC/C=C\CCCCCCCC(=O)OC[C@H](C[N+](C)(C)C)OC(=O)CCCCCCC/C=C\CCCCCCCC]

(DOTAP); and a nucleic acid therapeutic cargo encoding a gene for rescuing gene expression in a smooth muscle cell.

In some embodiments, the disclosure provides a particle comprising: a) an amount of an ionizable lipid; an amount of neutral lipid; an amount of cholesterol; and an amount of one or more PEG-lipids; an amount of a DOTAP molecule; and b) a nucleic acid therapeutic cargo encoding a gene for rescuing gene expression in a smooth muscle cell. In some embodiments, the particle comprises amounts of the ionizable lipid, the neutral lipid, the cholesterol, the one or more PEG-lipids, and DOTAP at a molar ratio of about 10:2.1:7.6:1.5:78.8.

Other features and advantages of the invention will be apparent from the following detailed description and figures, and from the claims.

### DESCRIPTION OF DRAWINGS

The foregoing and other features and advantages of the present invention will be more fully understood from the following detailed description of illustrative embodiments taken in conjunction with the accompanying drawings in which:

FIGS. 1A-1C show LNPs in vitro screening in liver hepatocytes cells (HepG2 cell line) based on formulation composition varying in cholesterol and DOPE content (high, medium, low). FIG. 1A is a graph showing the delivery of plasmid expressing RFP, measured by flow cytometry. FIGS. 1B-1C) are gel electrophoresis images showing the delivery of plasmid construct expressing soluble srENPP1 and detection at (FIG. 1B) cell lysates and (FIG. 1C) cell's supernatant.

FIG. 2 are microscopy images illustrating efficient soluble srENPP1 expression in the liver among other organs 6 days post injection with a particle of the disclosure in an Asj mouse model for generalized arterial calcification of infancy (GACI). Asj mice injected with soluble srENPP1 plasmid (0.3 mg/kg) at day 3 (P3) express high levels of the enzyme in the liver, 6 days post injection.

FIGS. 3A-3D show an efficacy study of LNP delivery using plasmids expressing soluble srENPP1. FIG. 3A is a schematic illustration presenting the injection regimen. FIG. 3AB are survival curves of treated animals, FIG. 3C is a graph showing animal body weight, and FIG. 3D are microCT scans to detect early development of calcification in treated (LNPs encapsulating soluble ENPP1, 0.3 mg/kg) and untreated animals.

FIGS. 4A-4B are schematic illustrations of LNP components and graphs showing the physical characterization of the LNPs. FIG. 4A is a schematic illustration presenting the LNP four-component system encapsulating plasmid DNA and the addition of a fifth lipid. FIG. 4B are graphs showing LNPs' hydrodynamic size, PDI, and zeta potential as a function of % of DOTAP in the formulation.

FIGS. 5A-5B are charts illustrating that introduction of DOTAP lipid into a 4-component formulation enables the efficient delivery of plasmid encoding red fluorescent protein (RFP) to MOVAS cells (a mouse vascular smooth muscle cell line). MOVAS cells were incubated for 48 h with plasmid (2 ug/48 well plate) encoding RFP. Cellular expression was identified and measured using flow cytometry. FIG. 5A is a graph depicting the % RFP positive cells as a function of % of DOTAP. FIG. 5B is a graph depicting the mean fluorescence intensity of RFP expression as a function of % of DOTAP.

FIGS. 6A-6F are histological images illustrating that the introduction of DOTAP LNPs enabled the in vivo delivery of plasmid DNA to smooth muscle cells (SMCs) at the aorta. In vivo transduction of transmembrane rENPP1 in SMCs at the aorta using DOTAP LNPs. Asj mouse model (mice that lack endogenous ENPP1) were injected at P3 systemically with PBS (control)(FIGS. 6A and 6D), LNPs (conventional four-component formulation)(FIGS. 6B and 6E) and DOTAP LNPs formulation (7% DOTAP)(FIGS. 6C and 6F) at 0.3 mg/kg plasmid dose. Histological images of the aorta in which SMCs (F-actin)(FIGS. 6D, 6E, and 6F) colocalize with ENPP1 expression (Flag-tag)(FIGS. 6A, 6B, and 6C). The DOTAP LNPs exhibit improved smooth muscle cell transduction compared to LNPs in vivo.

FIGS. 7A-7B are schematic illustrations of LNP components and graphs showing the physical characterization of the LNPs. FIG. 7A is a schematic illustration presenting the LNP four-component system encapsulating mRNA and the addition of a fifth lipid; FIG. 7AB are charts showing the LNPs' hydrodynamic size, PDI, and zeta potential as a function of % of DOTAP in the formulation.

FIGS. 8A-8F are graphs illustrating that introduction of DOTAP into the formulation enables the in vitro delivery of mRNA encoding GFP to MOVAS cell line. (FIGS. 8A and 8B) MOVAS cells incubated for 24 h with DOTAP/LNPs encapsulating mRNA (0.1 ug/48 well plate) encoding GFP. Cellular expression was identified and measured using flow cytometry presenting (FIG. 8A) percentage of cells expressing GFP and (FIG. 8B) GFP fluorescent intensity. (FIG. 8C) MOVAS cell viability, assessed to confirm the lack of treatment toxicity. Cellular expression of MOVAS cells was identified and measured using (FIG. 8D) fluorescent microscopy, based on GFP fluorescent intensity. (FIG. 8E) is a schematic illustration of primary human aortic ACTA2 mutant cells. (FIG. 8F) Primary human aortic cells cultured from ACTA2 patient were incubated with 10% DOTAP/LNPs encapsulating mRNA encoding GFP. Cellular expression of the primary human aortic cells was identified and measured using flow cytometry based on GFP fluorescent intensity.

FIGS. 9A-9C show a schematic illustration of a cellular uptake study (FIG. 9A) of 10% DOTAP LNPs in comparison to 0% and 100% determined by (FIG. 9B) fluorescent microscopy and (FIG. 9C) flow cytometry at 4 hour and 24 hour timepoints. LNPs encapsulated mRNA labeled by Cy5.

FIG. 10 depicts histological images of Cre-mRNA delivery utilizing LNPs with increasing DOTAP content and expression of tdTom in a Marfan disease mouse model. Increasing the % of DOTAP in LNP formulation increases SMC tdTom expression in vivo. Mice at P3 injected with 1 mg/kg mRNA encapsulated in LNPs formulated with 0, 10, 50 and 80% DOTAP lipid. tdTom expression identified using immunofluorescence in histological sections of the aorta and localized to SMCs (indicated by α-SMA expression).

FIG. 11 is a schematic illustration presenting the LNPs' conjugation scheme. LNPs are conjugated with the desired peptide following the assembly of the nanoparticle. Peptide conjugation was done in different densities controlled by the percentage of the linker lipid in the LNP formulation that was 0.15-1.2%.

FIG. 12 is a chart depicting the correlation between percentage of linker lipid within peptide-conjugated LNPs, their measured peptide concentration and the resulting calculated number of peptides per LNP.

FIG. 13 is a chart depicting the functional activity of peptide-conjugated LNPs in vitro. MOVAS cells treated (24 h) by LNPs conjugated with Col-IV/IL6R/CD63/Gal3 targeting peptides encapsulating mRNA encoding GFP. Cellular expression was identified and measured using flow cytometry.

FIG. 14 are histological images depicting that Col-4 peptide conjugation enhances LNP targeting to SMCs, as demonstrated by tdTom expression in vivo. Cre-mRNA encapsulated in Col-4 peptide-conjugated LNPs was injected into Marfan disease mouse models at P3 at a dose of 1 mg/kg mRNA. Peptide surface density varied at 0.15%, 0.3%, and 0.6%, based on its relative percentage in the total LNP formulation. Six days post-injection, tdTom expression was assessed via immunofluorescence in histological sections of the aorta, revealing localization within smooth muscle cells (SMCs), identified by α-SMA staining.

FIG. 15 are histological images depicting that leveraging two approaches to target SMCs doesn't demonstrate an increase in SMC tdTom expression. LNPs composed of the 80% DOTAP formulation were conjugated by Col-IV peptide and compared to each of the counterparts' controls that were found most effective as a single approach (80% DOTAP formulation or 0.3%-Col-IV conjugated LNP). In addition, the effect of % of Col-IV peptide decoration on the 80% DOTAP formulation was examined to verify optimal combination. Cre-mRNA encapsulated in each of the LNPs was injected into Marfan disease mouse models at P3 at a dose of 1 mg/kg mRNA. Six days post-injection, tdTom expression was assessed via immunofluorescence in histological sections of the aorta, revealing localization within smooth muscle cells (SMCs), identified by α-SMA staining.

FIGS. 16A-16C are histological images indicating that a combination of Col-IV targeting peptide and IL-6R targeting peptide enhances gene expression and inflamed tissue targeting. LNPs encapsulating GFP mRNA conjugated by IL-6R targeting peptide (0.15%) and Col-IV targeting peptide (0.15%) intravenously administered to Myhre syndrome and wildtype (WT) mice. 48 h post injection organs were collected, and histology analysis is presented showing the extent of GFP expression and localization in VSMCs. Images present histology sections of (FIG. 16A) aorta, (FIG. 16B) liver and (FIG. 16C) kidneys.

FIG. 17 are histological images indicating that using IL-6R targeting peptide enhances gene expression and inflamed tissue targeting. LNPs encapsulating GFP mRNA conjugated by IL-6R targeting peptide (0.3%) and administered intravenously to LDLR knockout mice. 12 h post injection organs were collected and histology analysis was done to detect GFP expression in the aorta. The expression was compared to a non-conjugated LNPs holding the same lipid composition as the conjugated.

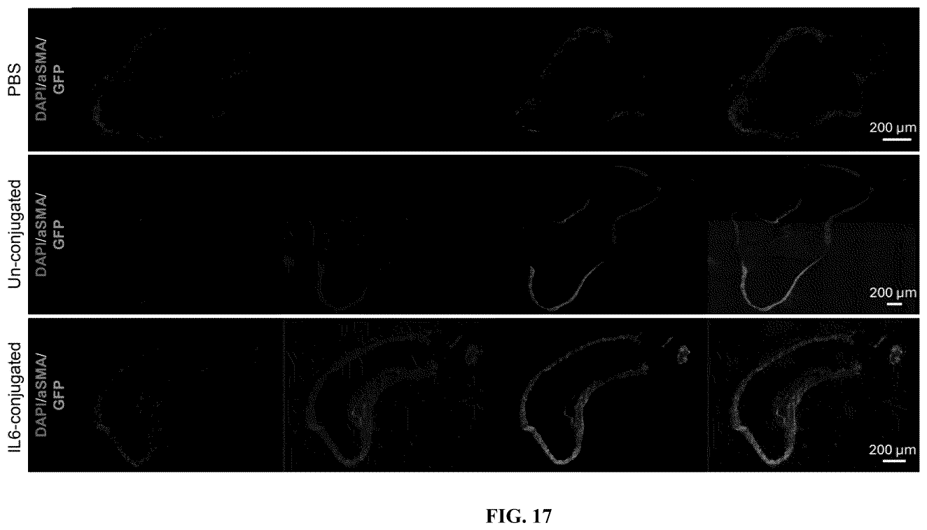

It should be understood that the drawings are not necessarily to scale (e.g., schematics), and that, when present, like reference numbers refer to like features.

### DETAILED DESCRIPTION

Provided herein are lipid nanoparticles (LNPs) and strategies for active delivery of target nucleic acids to smooth muscle cells (SMCs), in particular to vascular smooth muscle cells (vSMCs). Passive targeting of LNPs in the body is believed to be governed primarily by the size and charge of the LNP, which is acquired through changes in the molar compositions of the four types of lipids used in the formulation. In some aspects, the instant disclosure provides for lipid nanoparticles that comprise a permanently cationic lipid, in addition to having cholesterol, helper lipid(s), PEGylated lipid(s), and ionizable amine-containing lipid(s). The present disclosure demonstrates that certain ranges of permanently cationic lipids in formulations provide for particles and formulations with preferred tropism toward SMCs, in particular vSMCs. In some instances, such particles and formulations comprise from 0.1% to 80% (molar percentage) of 1,2-Dioleoyl-3-trimethylammonium propane (often abbreviated DOTAP or 18:1TAP), a di-chain, or gemini, cationic surfactant molecule of formula I.

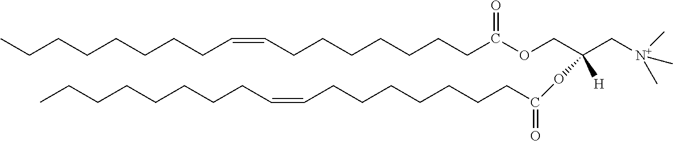 [SMILES: CCCCCCCC/C=C\CCCCCCCC(=O)OC[C@H](C[N+](C)(C)C)OC(=O)CCCCCCC/C=C\CCCCCCCC]

In some embodiments, the particles and formulations comprise from 0.1% to 80% (e.g., about 10% to about 80%) (molar percentage) of 1,2-Dioleoyl-3-trimethylammonium propane (often abbreviated DOTAP or 18:1TAP), a di-chain, or gemini, cationic surfactant molecule of formula I. In some aspects, provided herein are lipid nanoparticles comprising certain amounts of DOTAP, an ionizable lipid, a neutral lipid, cholesterol, and one or more PEG-lipids with demonstrable tropism towards smooth muscle cells. In some instances, the particles comprise an ionizable lipid, a neutral lipid, cholesterol, and one or more PEG-lipids at a molar ratio of about 50/10/38.5/1.5. In some instances, the particles comprise an ionizable lipid, a neutral lipid, cholesterol, and one or more PEG-lipids at a molar ratio of about 10/2.1/7.6/1.5. In many instances, the percentage of the ionizable lipid, the neutral lipid (e.g., phospholipid), the cholesterol, and one or more PEG-lipids in a particle is selected to accommodate the incorporation of DOTAP into the particle. Specifically, in instances where the amounts of DOTAP in a particle are selected to range from 0.1% to 80% (molar percentage) (e.g., about 0.1% to about 1%, about 0.1% to about 30%, about 0.1% to about 50%, about 0.1% to about 60%, about 0.1% to about 70%, about 0.1% to about 80%) of the total amounts of the ionizable lipid, the neutral lipid (e.g., phospholipid), the cholesterol, and the one or more PEG-lipids in the particles are adjusted to conform with the amounts of DOTAP in the particle.

For example, the amounts of the lipids, other than DOTAP, in the particle can be adjusted as follows: amounts of ionizable lipid can be adjusted to range from 5%, 6%, 7%, 8%, 9%, 10%, 11%, 12%, 13%, 14%, 15%, 45%, 46%, 47%, 48%, 49%, 50%, 51%, up to 52% (molar percentage); amounts of a neutral lipid can be adjusted to range from 1%, 2%, 3%, 9%, 10%, 11% (molar percentage), amounts of cholesterol can be adjusted to range from 5%, 6%, 7%, 8%, 9%, 10%, 11%, 30%, 31%, 32%, 33%, 34%, 35%, 36%, 37%, 38%, 39%, up to 40% (molar percentage); and amounts of the one or more PEG-lipids can be adjusted from 0.1%, 1%, 1.5%, up to 2% (molar percentage) (e.g., about 0.1% to about 0.15%, about 0.1% to about 0.3%, about 0.1% to about 0.6%, about 0.1% to about 0.9%, about 0.1% to about 1.2%, about 0.1% to about 1.4%, about 0.1% to about 2%, about 0.15% to about 0.3%, about 0.15% to about 0.6%, about 0.15% to about 0.9%, about 0.15% to about 1.2%, about 0.15% to about 1.4%, about 0.15% to about 2%, about 0.3% to about 0.6%, about 0.3% to about 0.9%, about 0.3% to about 1.2%, about 0.3% to about 1.4%, about 0.3% to about 2%, about 0.6% to about 0.9%, about 0.6% to about 1.2%, about 0.6% to about 1.4%, about 0.6% to about 2%, about 0.9% to about 1.2%, about 0.9% to about 1.4%, about 0.9% to about 2%, about 1.2% to about 1.4%, about 1.2% to about 2%, about 0.1% to about 1.5%, about 0.2% to about 1.5%, about 0.3% to about 1.5%, about 0.4% to about 1.5%, about 0.5% to about 1.5%, about 0.6% to about 1.5%, about 0.7% to about 1.5%, about 0.8% to about 1.5%, about 0.9% to about 1.5%, about 1% to about 1.5%, about 1.1% to about 1.5%, about 1.2% to about 1.5%, about 1.3% to about 1.5%, about 1.4% to about 1.5%, 1.5% to about 1.6%, 1.5% to about 1.7%, 1.5% to about 1.8%, 1.5% to about 1.9%, or 1.5% to about 2%) of the total amounts of lipids (% of total lipids) in the particle.

In some embodiments, the LNPs include about 80% (molar percentage) of DOTAP. In some embodiments, the LNPs include about 78.8% of DOTAP. In some embodiments, the LNPs include about 75% to about 85% (e.g., about 75% to about 78.8%, about 75% to about 79%, about 75% to about 80%, about 75% to about 81%, about 75% to about 82%, about 75% to about 83%, about 75% to about 84%, about 75% to about 85%, about 76% to about 78.8%, about 76% to about 79%, about 76% to about 80%, about 76% to about 81%, about 76% to about 82%, about 76% to about 83%, about 76% to about 84%, about 76% to about 85%, about 77% to about 78.8%, about 77% to about 79%, about 77% to about 80%, about 77% to about 81%, about 77% to about 82%, about 77% to about 83%, about 77% to about 84%, about 77% to about 85%, about 78% to about 78.8%, about 78% to about 79%, about 78% to about 80%, about 78% to about 81%, about 78% to about 82%, about 78% to about 83%, about 78% to about 84%, about 78% to about 85%, about 79% to about 80%, about 79% to about 81%, about 79% to about 82%, about 79% to about 83%, about 79% to about 84%, about 79% to about 85%, about 80% to about 81%, about 80% to about 82%, about 80% to about 83%, about 80% to about 84%, or about 80% to about 85%) of DOTAP. In some embodiments, the LNPs include about 0.1% to about 10% DOTAP.

In some embodiments, the LNPs include about 10% of an ionizable lipid. In some embodiments, the LNPs include about 5% to about 15% (e.g., about 6% to about 10%, about 7% to about 10%, about 8% to about 10%, about 9% to about 10%, about 10% to about 11%, about 10% to about 12%, about 10% to about 13%, about 10% to about 14%, or about 10% to about 15%) of an ionizable lipid.

In some embodiments, the LNPs include about 2.1% of a neutral lipid. In some embodiments, the LNPs include about 2% of a neutral lipid. In some embodiments, the LNPs include about 0.5% to about 3.5% (e.g., about 0.5% to about 2.1%, about 0.6% to about 2.1%, about 0.7% to about 2.1%, about 0.8% to about 2.1%, about 0.9% to about 2.1%, about 1% to about 2.1%, about 1.1% to about 2.1%, about 1.2% to about 2.1%, about 1.3% to about 2.1%, about 1.4% to about 2.1%, about 1.5% to about 2.1%, about 1.6% to about 2.1%, about 1.7% to about 2.1%, about 1.8% to about 2.1%, about 1.9% to about 2.1%, about 2% to about 2.1%, about 2.1% to about 2.2%, about 2.1% to about 2.3%, about 2.1% to about 2.4%, about 2.1% to about 2.5%, about 2.1% to about 2.6%, about 2.1% to about 2.7%, about 2.1% to about 2.8%, about 2.1% to about 2.9%, about 2.1% to about 3.0%, about 2.1% to about 3.1%, about 2.1% to about 3.2%, about 2.1% to about 3.3%, about 2.1% to about 3.4%, or about 2.1% to about 3.5%) of a neutral lipid.

In some embodiments, the LNPs include about 7.6% of cholesterol. In some embodiments, the LNPs include about 7% to about 8% of a neutral lipid. In some embodiments, the LNPs include about 5% to about 10% (e.g., about 5% to about 7.6%, about 6% to about 7.6%, about 7% to about 7.6%, about 7% to about 8%, about 7% to about 9%, about 7% to about 10%, about 7.6% to about 8%, about 7.6% to about 9%, about 7.6% to about 10%) of a neutral lipid.

In some embodiments, the LNPs include one or more PEG-lipids comprising DMG-PEG and DSPE-PEG-maleimide (DSPE-PEG-mal). In some embodiments, the LNPs include about 1.2% DMG-PEG and about 0.3% DSPE-PEG-mal. In some embodiments, the LNPs include about 0.3% to about 1.2% of DMG-PEG and about 0% to about 0.6% DSPE-PEG-mal. In some embodiments, the LNPs include about 0% to about 1.5% (e.g., about 0% to about 0.3%, about 0.3% to about 0.75%, about 0.3% to about 1%, about 0.3% to about 1.05%, about 0.3% to about 1.2%, about 0.3% to about 1.5%) of DMG-PEG. In some embodiments, the LNPs include about 0% to about 1% (e.g., about 0% to about 0.3%, about 0% to about 0.4%, about 0% to about 0.5%, about 0% to about 0.6%, about 0.1% to about 0.3%, about 0.1% to about 0.4%, about 0.1% to about 0.5%, about 0.1% to about 0.6%, about 0.2% to about 0.3%, about 0.2% to about 0.4%, about 0.2% to about 0.5%, about 0.2% to about 0.6%, about 0.3% to about 0.4%, about 0.3% to about 0.5%, about 0.3% to about 0.6%, about 0.3% to about 1%, or about 0.6% to about 1%) of DSPE-PEG-mal.

Specifically, amounts of DOTAP in a particle of the disclosure can be specified in terms of total lipid percentage. Specifically, in instances where the percentage of DOTAP in a particle is selected to range from about 0.1% to about 80% of the total percentage of lipids in the particles, the amounts of the other lipids in the particle can be adjusted based on the remaining lipid percentage as follows: amounts of ionizable lipid can be adjusted to range from 10% up to 52% of the remaining lipid percentage; amounts of a neutral lipid can be adjusted to range from 2% up to 11% of the remaining lipid percentage, amounts of cholesterol can be adjusted to range from 7% up to 40% of the remaining lipid percentage; and amounts of one or more PEG-lipids can be adjusted from 0% up to 2% of the remaining lipid percentage.

For example, when up to 10% DOTAP is added, the remaining 90% of the total lipid amount is distributed accordingly among the remaining lipids. For example, in some embodiments, if the percentage of DOTAP in a particle is 10%, the amounts of the other lipids in the particle can be adjusted based on the remaining 90% as follows: amounts of ionizable lipid can be adjusted to range from 45% up to 52% of the remaining 90%; amounts of a neutral lipid can be adjusted to range from 9% up to 11% of the remaining 90%, amounts of cholesterol can be adjusted to range from 34% up to 40% of the remaining 90%; and amounts of one or mores PEG-lipids can be adjusted from 0.1% up to 2% of the remaining 90% of the total lipids in the composition.

In another example, when up to 80% DOTAP is added, the remaining 10% of the total lipid amount is distributed accordingly among the remaining lipids. For example, in some embodiments, if the percentage of DOTAP in a particle is 80%, the amounts of the other lipids in the particle can be adjusted based on the remaining 10% as follows: amount of ionizable lipid can be adjusted to range from 7% up to 13% of the remaining 10%; amount of a neutral lipid can be adjusted to range from 1% up to 3% of the remaining 10%, amount of cholesterol can be adjusted to range from 6% up to 8% of the remaining 10%; and amounts of the one or more PEG-lipids can be adjusted from 0.1% up to 2% of the remaining 10% of the total lipids in the composition.

In some instances, the disclosure provides a therapeutic formulation comprising: about 78.8% of DOTAP; about 10% of an MC3 ionizable lipid; about 2.1% of a DOPE neutral lipid; about 7.6% of cholesterol; and about 1.5% of one or more PEG-lipids (e.g., about 0.3% DSPE-PEG-mal and about 1.2% DMG-PEG).

In some instances, the disclosure provides a therapeutic formulation comprising: about 80% of DOTAP; about 10% of an MC3 ionizable lipid; about 2.1% of a DOPE neutral lipid; about 7.6% of cholesterol; and about 0.3% of one or more PEG-lipids (e.g., about 0.3% DMG-PEG).

In some instances, the disclosure provides a therapeutic formulation comprising: about 80% of DOTAP; about 10% of an MC3 ionizable lipid; about 2.1% of a DOPE neutral lipid; about 7.6% of cholesterol; and about 0.3% of one or more PEG-lipids (e.g., about 0.3% DSPE-PEG-mal).

In some instances, the disclosure provides a therapeutic formulation comprising: about 78.8% of DOTAP; about 10% of an MC3 ionizable lipid; about 2.1% of a DOPE neutral lipid; about 7.6% of cholesterol; and about 1.5% of one or more PEG-lipids (e.g., about 0.6% DSPE-PEG-mal and about 0.9% DMG-PEG).

In some embodiments, the particles comprise: about 75-85% of DOTAP; about 10% of an MC3 ionizable lipid; about 2-2.5% of a DOPE neutral lipid; about 7-8% of cholesterol; and about 1-2% of one or more PEG-lipids. In some embodiments, the particles comprise: about 78.8% of DOTAP; about 10% of an MC3 ionizable lipid; about 2.1% of a DOPE neutral lipid; about 7.6% of cholesterol; and about 1.5% of one or more PEG-lipids.

In some embodiments, the LNPs provided herein can be spherical or ellipsoidal, or can have an amorphous shape. In some embodiments, the LNPs provided herein (e.g., conjugated or non-conjugated LNPs) can have a diameter (between any two points on the exterior surface of the LNP) of between about 100 nanometers (nm) to about 250 nm (e.g., between about 100 nm to about 150 nm, between about 100 nm to about 200 nm, between about 100 nm to about 250 nm, between about 125 nm to about 150 nm, between about 150 nm to about 175 nm, between about 150 nm to about 200 nm, between about 150 nm to about 250 nm). In some embodiments, LNPs having a diameter of between about 100 nm to about 250 nm localize to the diseased vasculature in a subject. In some embodiments, LNPs having a diameter of between about 100 nm to about 150 nm localize to the smooth muscle cells of a subject.

### Lipid Nanoparticles (LNPs)

The LNP compositions of the disclosure can be prepared by various techniques which are presently known in the art. Multilamellar vesicles (MLVs) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULVs), such as the LNPs of the disclosure, can then be formed by homogenization, sonication, or extrusion of the multi-lamellar vesicles. In addition, unilamellar vesicles can be formed by detergent removal techniques.

In many instances, the particles, formulations, and compositions of the disclosure comprise at least the following five lipid components:

### Permanently Cationic Lipids

Structurally, synthetic and/or natural lipids usually contain three parts: (i) cationic or ionizable head groups, (ii) linker groups, and (iii) hydrophobic tails. The chemical diversity of each part results in a number of structurally distinct ionizable lipids that can be produced by combinatorial chemistry. Conventional permanently charged cationic lipids previously used for nucleic acid delivery (e.g., DOTAP) are believed to readily interact with negatively charged serum proteins and aggregate in the bloodstream, which was believed to lead to rapid clearance of LNP by mononuclear phagocytes. Thus, the relatively high hemolytic activity of cationic lipids was believed to increase the risk of toxic side effects, such as hemoglobin release due to red cell membrane damage.

The disclosure demonstrates that the presence of certain ratios or amounts of permanently cationic lipids (e.g., DOTAP) in a particle can preferably target the particle to vascular smooth blood cells (vSMCs), in vitro and in vivo. In some embodiments, the particles of the disclosure include DOTAP. In some embodiments, the particles of the disclosure include DOTAP, 1,2-di-O-octadecenyl-3-trimethylammonium propane (chloride salt) (DOTMA), dimethyldioctadecylammonium (DDAB), 1,2-dimyristoyl-sn-glycero-3-ethylphosphocholine (EPC), or any combination thereof.

### Ionizable Cationic Lipids

Ionizable cationic lipids are traditional components in many existing LNP formulation(s). Their acid dissociation constants (pKa) determine the ionization and surface charge of the LNP, further affecting its stability and toxicity. To avoid these problems, ionizable cationic lipids with pKa values typically ranging from 6.0 to 7.0 have been developed and deployed, most notably in vaccine formulations. This ionizable lipid-based LNP (iLNP) ensures efficient encapsulation of nucleic acids under acidic conditions and reduces toxicity during recycling under physiological conditions. After entering endosomes/lysosomes (which have a pH below surface pKa), LNPs can be positively charged again to facilitate endosome escape and release mRNA into the cytoplasm. It has been reported that LNPs with pKa values of 6.2-6.5 and 6.6-6.9 favored hepatic delivery of siRNA in vivo and intramuscular administration of mRNA vaccines, respectively.

Depending on the number of amino heads, ionizable cationic lipids can be classified as either monoamino or polyamino lipids. Non-limiting examples of monoamino acid ionizable cationic lipids contemplated in particles of the disclosure include DLin-MC3-DMA (MC3), SM-102, and ALC-0315. Non-limiting examples of monoamino acid ionizable cationic lipids contemplated in particles of the disclosure include 306Oᵢ₁₀, cKK-E12, C12-200, 5A2-SC8, TT3, and FTT5. In many instances, the ionizable lipid is selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DMA, DLin-K-C4-DMA, DLen-C2K-DMA, γ-DLen-C2K-DMA, or DLin-MP-DMA.

### Peg-Lipids

Although PEG-lipids generally constitute the smallest molar percentage of the lipid components in LNPs (typically about 0.5 mol % and up to about 2.0 mol %), they have several effects on the properties of lipid nanoparticles, including influencing particle size and zeta potential. A variety of PEG lipids are contemplated for use with the LNPs of the disclosure, including terminally modified PEG lipids.

PEG lipids for use in the present disclosure can be, for example, maleimide terminally modified PEG lipids that can be conjugated with cell targeting peptides. PEG lipids for use with the instant LNPs can have the general structure —(CH₂CH₂O)n- or —(CH₂CH₂O) nCH₂CH₂. This general structure can further be modified with heterobifunctional maleimide linker. The disclosure contemplates that a variety of PEG molecules can be incorporated into its LNPs, including poly(ethylene glycol) (PEG) maleimide (e.g., PEG-2000 maleimide), polyalkylene glycols, polypropylene or polybutylene glycols, methoxy poly (ethylene glycol), or methoxy poly (ethylene glycol) propionic acid (mPEG-acid) where n can be from about 1 to about 400. An LNP comprising a thiol reactive motive conjugated to a PEG molecule (e.g., heterobifunctional maleimide PEG) can be decorated with various types of peptides that are displayed on the surface of the LNP molecule.

In some instances, a PEG molecule is linked to a thiol reactive group for further conjugation to a peptide. In some instances the PEG molecule is a maleimide conjugated PEG molecule. Reactive PEGs can be used for amine pegylation, thiol pegylation, or N-terminal pegylation. The amine in the N-terminus and/or the carboxyl group in the C-terminus can react with a targeting peptide.

In some instances, a PEG-lipid of the one or more PEG-lipids that is suitable for use in the particles of the disclosure is 1,2-dimyristoyl-rac-glycero-3-methoxypolyethylene glycol (DMG-PEG). In some embodiments, a PEG-lipid of the one or more PEG-lipids that is suitable for use in the particles of the disclosure is DMG-PEG 2000. In some instances, the PEG molecule is methoxy poly (ethylene glycol) succinimidyl proprionate (mPEG-SPA). In some instances, a PEG molecule is a methoxy poly (ethylene glycol) propionic acid (mPEG-acid). In some cases, the polyethylene glycol molecule weighs from about 1,000 kilodaltons to about 5,000 kilodaltons. The covalent attachment of a targeting peptide to an LNP via a thiol reactive linkage can change the physicochemical characteristics of the LNP. Examples of physicochemical characteristics that can be altered by binding to a PEG include its zeta potential, its PDI, and the overall hydrodynamic size of the LNP.

Non-limiting examples of commercially available PEGs suitable for use in the particles of the disclosure include, but are not limited to those available from Nektar Therapeutics, San Carlos, CA, such as mPEG-NH2 (Mw about 10 kDa, about 20 kDa), methoxy PEG Succinimidyl α-Methylbutanoate (SMB), SMB-PEG-SMB, methoxy PEG Succinimidyl Propionate (mPEG-SPA), Branched PEG N-Hydroxysuccinimide (mPEG2-NHS), mPEG-CM-HBA-NHS, NHS-HBA-CM-PEG-CM-HBA-NHS, mPEG-ButyrALD, ButyrALD-PEG-ButyrALD, Branched PEG ButyrALD (mPEG2-ButyrALD), Ortho-pyridylthioester (mPEG-OPTE), mPEG Maleimide (MAL), MAL-PEG-MAL, Branched PEG Maleimide (mPEG2-MAL), Forked Maleimide (mPEG-MAL2 and mPEG2-MAL2), mPEG-Ortho-pyridyldisulfide (mPEG-OPSS), OPSS-PEG-OPSS, mPEG-SH, SH-PEG-SH, Amine-PEG-Acid, Boc-PEG-NHS, Fmoc-PEG-NHS, MAL-PEG-NHS, Vinylsulfone-PEG-NHS, Acrylate-PEG-NHS Ester.

Non-limiting examples of PEGs that can be used in amine pegylation include, for example, PEGs manufactured by Jenken Technology USA such as: Y-shape PEG NHS Esters, Y-shape PEG Carboxyl, Glucose PEG NHS Ester, Galactose PEG NHS Ester, Methoxy PEG Succinimidyl Carboxymethyl Ester, Methoxy PEG Carboxyl, Methoxy PEG Succinimidyl Butanoate, Methoxy PEG Succinimidyl Hexanoate, Methoxy PEG Hexanoic Acid, Methoxy PEG Succinimidyl Succinamide, Methoxy PEG Succinimidyl Glutaramide, Methoxy PEG Succinimidyl Carbonate, Methoxy PEG Nitrophenyl Carbonate, Methoxy PEG Succinimidyl Succinate, Methoxy PEG Succinimidyl Glutarate. Non-limiting examples of PEGs that can be used in thiol pegylation include Y-shape PEG Maleimide, Methoxy PEG Maleimide, Methoxy PEG Vinylsulfone, Methoxy PEG Thiol. Non-limiting examples of PEGs that can be used in N-terminal pegylation include, for example, PEGs manufactured by Jenken Technology USA such as: Y-shape PEG Aldehyde, Y-shape PEG Acetaldehyde, Y-shape PEG Propionaldehyde, Methoxy PEG Propionaldehyde.

In many instances, a targeting peptide can have a molecular weight that is small compared to the PEG molecule to which it is attached. The molecular weight of a PEG molecule used in an LNP of the disclosure can be, for example, no greater than 5 kilodaltons (kDa), no greater than 4.5 kilodaltons, no greater than 4 kilodaltons, no greater 3.5 than kilodaltons (kDa), no greater than 3 kilodaltons (kDa), no greater than 2.5 kilodaltons (kDa), no greater than 2 kilodaltons (kDa), no greater than 1.5 kilodaltons (kDa), or no greater than 1 kilodaltons (kDa).

In some cases, the molecular weight of a PEG molecule can be greater than 1 kilodalton (kDa), greater than 1.5 kilodaltons (kDa), greater than 2 kilodaltons (kDa), greater than 2.5 kilodaltons (kDa), greater than 3 kilodaltons (kDa), greater than 3.5 kilodaltons (kDa), greater than 4 kilodaltons (kDa), or greater than 4.5 kilodaltons (kDa).

In some cases the molecular weight of a PEG oligomer can be from about 1 kilodalton (kDa) to about 5 kilodaltons (kDa), from about 1 kilodalton (kDa) to about 2 kilodaltons (kDa), from about 1 kilodaltons (kDa) to about 3 kilodaltons (kDa), from about 1 kilodaltons (kDa) to about 4 kilodaltons (kDa), from about 1 kilodaltons (kDa) to about 5 kilodaltons (kDa), from about 1.5 kilodaltons (kDa) to about 2 kilodaltons (kDa), from about 1.5 kilodaltons (kDa) to about 3 kilodaltons (kDa), from about 1.5 kilodaltons (kDa) to about 3.5 kilodaltons (kDa), from about 1.5 kilodaltons (kDa) to about 4 kilodaltons (kDa), from about 1.5 kilodaltons (kDa) to about 4.5 kilodaltons (kDa), from about 1.5 kilodaltons (kDa) to about 5 kilodaltons (kDa), from about 2 kilodaltons (kDa) to about 3 kilodaltons (kDa), from about 2 kilodaltons (kDa) to about 3.5 kilodaltons (kDa), from about 2 kilodaltons (kDa) to about 4 kilodaltons (kDa), from about 2 kilodaltons (kDa) to about 4.5 kilodaltons (kDa), from about 2 kilodaltons (kDa) to about 5 kilodaltons (kDa).

In some embodiments, the molecular weight of a maleimide-terminally modified PEG lipid is about 2 kilodaltons (kDa). In some embodiments, the molecular weight of a PEG molecule is from about 1 kilodaltons (kDa) to about 5 kilodaltons (kDa).

### Neutral-Lipids Helper Lipids—Phospholipids

Phospholipids are neutral “helper” lipids that contribute to the formation of lipid nanoparticles and the escape of endosomes. In many instances, a particle of the disclosure comprises a neutral lipid that is a phosphatidylcholine lipid or a phosphatidylethanolamine lipid. The phosphatidylcholine lipid or the phosphatidylethanolamine lipid can be selected from the group comprising 1,2-dioleoyl-sn-glycero-3-phosphoethanolamine (DOPE), 1,2-dioleoyl-sn-glycero-3-phosphocholine (DOPC), 1,2-distearoyl-sn-glycero-3-phosphocholine (DSPC), 1,2-dipalmitoyl-sn-glycero-3-phosphocholine (DPPC), 1-palmitoyl-2-oleoyl-glycero-3-phosphocholine (POPC), and 1-stearoyl-2-oleoyl-sn-glycero-3-phosphocholine (SOPC).

### Cholesterol

The inclusion of cholesterol in nucleic acid-containing LNP formulations is based primarily on two major findings obtained with liposomal formulations of small molecule therapeutics: 1) cholesterol is an exchangeable molecule that can accumulate within liposomes during circulation, 2) cholesterol dramatically reduces the amount of surface-bound proteins and improves the circulating half-life.

### Therapeutic Cargos

In some aspects, the disclosure is based on particles and compositions comprising such particles for delivering a therapeutic cargo to a smooth muscle cell. In many instances, the therapeutic cargo is one or both of an mRNA molecule and/or a plasmid encoding a transgene, optionally a transgene for rescuing gene expression in a cell or subject that is administered the particle. In some aspects, such particles display tropism towards smooth muscle cell, in particular vascular smooth muscle cells.

In some aspects, the therapeutic cargo is a nucleic acid molecule encoding an ectonucleotide pyrophosphatase/phosphodiesterase 1 (ENPP1) transgene. In some embodiments, the ENPP1 therapeutic cargo comprises gene therapy constructs for expression of ENPP1, e.g., soluble recombinant ENPP1 (srENPP1) or transmembrane recombinant ENPP1 (rENPP1), in cells of a subject. The constructs can thus include sequences encoding full-length human ENPP1 or a truncated version thereof comprising the extracellular domain of human ENPP1 (srENPP1). The constructs can optionally be codon optimized. Exemplary sequences of human ENPP1 protein are provided in GenBank at RefSeq ID NM_006208.3 (nucleic acid) and NP_006199.2 (protein), e.g., as follows:

| (SEQ ID NO: 1) | |
|---|---|
| 1 | merdgcaggg srggeggrap regpagngrd rgrshaaeap gdpqaaasll apmdvgeepl |
| 61 | ekaarartak dpntykvlsl vlsvcvltti lgcifglkps cakevksckg rcfertfgnc |
| 121 | rcdaacvelg nccldyqetc iepehiwtcn kfrcgekrlt rslcacsddc kdkgdcciny |
| 181 | ssvcqgeksw veepcesine pqcpagfetp ptllfsldgf raeylhtwgg llpvisklkk |
| 241 | cgtytknmrp vyptktfpnh ysivtglype shgiidnkmy dpkmnasfsl kskekfnpew |
| 301 | ykgepiwvta kyqglksgtf fwpgsdvein gifpdiykmy ngsvpfeeri lavlqwlqlp |
| 361 | kderphfytl yleepdssgh sygpvssevi kalqrvdgmv gmlmdglkel nlhrclnlil |
| 421 | isdhgmeqgs ckkyiylnky lgdvknikvi ygpaarlrps dvpdkyysfn yegiarnlsc |
| 481 | repnqhfkpy lkhflpkrlh faksdriepl tfyldpqwql alnpserkyc gsgfhgsdnv |
| 541 | fsnmqalfvg ygpgfkhgie adtfenievy nlmcdlinlt papnngthgs lnhllknpvy |
| 601 | tpkhpkevhp lvqcpftrnp rdnlgcscnp silpiedfqt qfnltvaeek iikhetlpyg |
| 661 | rprvlqkent icllsqhqfm sgysqdilmp lwtsytvdrn dsfstedfsn clyqdfripl |
| 721 | spvhkcsfyk nntkvsygfl sppqlnknss giysealltt nivpmyqsfq viwryfhdtl |
| 781 | lrkyaeerng vnvvsgpvfd fdydgrcdsl enlrqkrrvi rnqeilipth ffivltsckd |
| 841 | tsqtplhcen ldtlafilph rtdnsescvh gkhdsswvee llmlhrarit dvehitglsf |
| 901 | yqqrkepvsd ilklkthlpt fsqed |

In some embodiments, the srENPP1 sequence comprises amino acids 103-925, and optionally amino acids 96-925 or 97-925.

In some aspects, the therapeutic cargo is a nucleic acid molecule encoding an actin alpha 2, smooth muscle (ACTA2) transgene or ATP Binding Cassette Subfamily C Member 6 (ABCC6). The following Table 1 provides accession numbers for exemplary sequence for human ACTA2 and ABCC6.

TABLE 1
Exemplary sequences for human ACTA2 (actin
alpha 2, smooth muscle) and ABCC6

| Nucleic Acid | | | |
|---|---|---|---|
| Molecule | Transcript | Protein | Isoform |
| ACTA2 | NM_001406467.1 | NP_001393396.1 | 3 |
| ACTA2 | NM_001320855.2 | NP_001307784.1 | 1 |
| ACTA2 | NM_001406462.1 | NP_001393391.1 | 1 |
| ACTA2 | NM_001406468.1 | NP_001393397.1 | 3 |
| ACTA2 | NM_001406471.1 | NP_001393400.1 | 4 |
| ACTA2 | NM_001141945.3 | NP_001135417.1 | 1 |
| ACTA2 | NM_001406463.1 | NP_001393392.1 | 1 |
| ACTA2 | NM_001406469.1 | NP_001393398.1 | 3 |
| ACTA2 | NM_001406466.1 | NP_001393395.1 | 2 |
| ACTA2 | NM_001613.4 | NP_001604.1 | 1 |
| ACTA2 | NM_001406464.1 | NP_001393393.1 | 1 |
| ABCC6 | NM_001171.6 | NP_001162.5 | 1 |
| ABCC6 | NM_001079528.4 | NP_001072996.1 | 2 |
| ABCC6 | NM_001351800.1 | NP_001338729.1 | 3 |

In some aspects, the therapeutic cargo is a nucleic acid molecule (e.g., plasmid or mRNA) encoding a CRISPR-Cas genome editing protein, e.g., a genome editor or an intein-split construct thereof, optionally selected from CRISPR nucleases, cytosine base editors (CBEs), adenine base editors (ABEs), and CRISPR prime editors (PEs), and variants thereof, as well as guide RNAs (gRNAs) that direct the editor to a target site in the genome (or sequences encoding such gRNAs). Genome editing technologies enable the permanent modification of DNA sequences in living cells. This process has been simplified by the discovery that CRISPR nucleases such as Cas9 can be readily programmed to edit DNA sites using a guide RNA (gRNA). Once the Cas enzyme is complexed with the gRNA, the Cas9-gRNA ribonucleoprotein (RNP) molecule scans chromosomal sequences for a short protospacer-adjacent motif (PAM) directly beside the target site. The simplicity of re-targeting the Cas9 protein to new sites offers the ability to make precise changes to the genome. However, the requirement for the Cas9 protein to recognize a PAM fundamentally limits the breadth of editing. Recently developed CRISPR-Cas9 and -Cas12a proteins with vastly expanded targeting ranges permit editing of previously inaccessible sequences can overcome this major limitation. Beyond traditional nuclease-based editing, cytosine base editors (CBEs) and adenine base editors (ABEs) have been recently developed that enable the introduction of precise C-to-T and A-to-G changes, respectively. Additionally, CRISPR prime editors (PEs) permit the installation of custom changes into the genome by using a reverse transcriptase (RT) domain and a prime editor guide RNA (pegRNA). Thus, a number of options exist for using CRISPR-Cas enzymes for precise modelling or correction of disease-causing mutations. However, not all of these options are equally successful in generating specific changes with minimal off-target effects. Together, the continually expanding toolbox of CRISPR-Cas enzymes offers technologies that enable the precise modelling or correction of disease-causing mutations.

An exemplary base editor is described in WO2024073715, for correction of the R179H mutation in the human ACTA2 gene, and comprises an adenine base editor agent can include a base editor, e.g., a base editor comprising a catalytically dead Cas9 (dCas9) or a nickase Cas9 (nCas9) fused to a deaminase and guided by a single guide RNA (sgRNA) to a sequence of interest. In some embodiments, the deaminase comprises an engineered adenosine deaminase TadA monomer or dimer comprises a homodimeric or heterodimeric TadA domain from ABEmax, ABE7.10, or ABE8e; monomer or dimer TadA from ABE 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, ABEmax, ABE8.8, ABE8.13, ABE8.17, ABE8.20, ABE8e, ABE9, ABE9e, or K20A/R21A, V82G, or V106W variants thereof, E. coli TadA monomer, or homo- or heterodimers thereof fused to the N or C terminus, optionally comprising one or more mutations in either or both monomers, optionally TadA from miniABEmax-V82G, miniABEmax-K20A/R21A, miniABEmax-V106W, or another variant. In some embodiments, the Cas9 portion of the base editor comprises the D1135L/S1136W/G1218K/E1219Q/R1335Q/T1337R (SpG; see, e.g., WO 2021/151073), D1135V/G1218R/R1335Q/T1337R (VRQR; see, e.g., WO 2016/141224), S55R/D1135V/G1218R/R1335Q/T1337R (VRQR(S55R), also referred to as VRQR+), A61R/L1111R/D1135L/S1136W/G1218K/E1219Q/N1317R/A1322R/R1333P/R1335 Q/T1337R (SpRY; see, e.g., WO 2021/151073), D10T(optional)/I322V/S409I/E427G/R654L/R753G/R1114G/D1135N/V1139A/D1180G/E1219V/Q1221H/A1320V/R1333K (SpCas9-NRRH), D10T(optional)/I322V/S409I/E427G/R654L/R753G/R1114G/D1135N/E1219V/D1332N/R1335Q/T1337N/S1338T/H1349R (SpCas9-NRCH), D1135M/S1136Q/G1218K/E1219S/R1335E/T1337R (MQSKER; see, e.g., US20210261932) variants of SpCas9. In some embodiments, the methods use the HES1208-A4 gRNA (with an NGA PAM and the target adenine in position A4 of the spacer, optionally with ABE8e-SpCas9-VRQR, ABE8e-SpCas9-VRQR(S55R), ABE8e-SpG, ABE8e-SpRY, or ABE8e-SpCas9-NRRH; Table A), or the HES1210-A7 gRNA (with a GGC PAM and the target adenine in position A7 of the spacer, optionally with ABE8e-SpG, ABE8e-SpRY, ABE8e-SpCas9-NRCH, ABE8e-MQSKER; Table A); or HES1212-A8 (with a TGG PAM and the target adenine in position A8 of the spacer, optionally with ABE8e-SpCas9, ABE8e-SpG, ABE8e-SpRY, or ABE8e-SpCas9-NRRH; Table A), provided an excellent combination of on-target editing activity without significant off-target or bystander base editing.

For allele-specific deletion of mutant alleles, genome editors, e.g., nucleases including those listed in Table B, guided by a single guide RNA (sgRNA) to a sequence of interest, can be used. For example, wild-type SpCas9, optionally with the HES1236 or HES1235 gRNAs (Table B), can be used to selectively knock-out the mutant R179H allele.

In any of the methods or compositions described herein, the base editor or genome editor can be delivered as an intein-split construct, e.g., as described in Levy et al., Nat Biomed Eng 4, 97-110 (2020), WO2016112242, Truong et al., Nucleic Acids Res. 2015 Jul. 27; 43(13):6450-8; and Yuan et al., ACS Synth. Biol. 2022, 11, 7, 2513-2517.

TABLE A
Target sites across ACTA2-R179H for base editing

| | | SEQ | | | | | |
|---|---|---|---|---|---|---|---|
| | Target site | ID | | Target | Target | | |
| Target site | with PAM | NO: | GANA | nt | site | | |
| name | (R179H bold) | | ID | pos'n | length | PAM | Use with enzymes: |
| human-ACTA2- | CATCATGCATCTGGA | 55. | | AS | 23 | CTG | ABE8e-SpRY |
| BE-NIG-SpCas9- | TCTGGCTG | | | | | | |
| A9 | | | | | | | |
| human-ACTA2- | ATCATGCATCTGGAT | 56. | HES1212 | A8 | 23 | TGG | ABE8e-SpCas9, ABE8e-SpG, ABBBe-SpRY, |
| ES-NGG-SpCas9- | CTGGCTGG | | | | | | ABE8e-SpCas9-NRRH |
| AB | | | | | | | |
| human-ACTA2- | TCATGCATCTGGATC | 57. | HES1210 | A7 | 23 | GGC | ABRBe-SpG, ABE8e-SpXX, ABE88-SpCas9- |
| BE-NGC-SpCas9- | TGGCTGGC | | | | | | NRCA |
| A? | | | | | | | |
| human-ACTA2- | CATGCATCTGGATCT | 58. | | A6 | 23 | GCC | ABE3e-SpRY |
| BE-NCC-SpCas9- | GGCTGGCC | | | | | | |
| A6 | | | | | | | |
| human-ACTA2- | ATGCATCTGGATCTG | 59. | | A5 | 23 | CCG | ABE8e-SpRY |
| BB-NCG-SpCas9- | GCTGGCCG | | | | | | |
| 35 | | | | | | | |
| human-ACTA2- | TGCATCTGGATCTGG | 60. | HES1208 | A4 | 23 | CGA | ABE8e -SpCas9-VRQR, ABE8e-SpCas9- |
| BE-NGA-SpCas9- | CTGGCCGA | | | | | | VRQR (855R), ABE8e-SpG, ABE86-SpRY, |
| A4 | | | | | | | ABE86-SpCas9-NRRB |
| mouse-ACTA2- | TGCATCTGGACTTGG | 61. | HES1370 | A4 | 23 | CGA | ABE8e-SpCas9-VRQR, ABBBe-SpCas9- |
| BE-NGA-SpCas9- | CTGGCCGA | | | | | | VRQR (SBSR), ABE8e-SpG, ABE8e-SpRY, |
| A4 | | | | | | | ABE8e-SpCas9-NRRH |
| human-ACTA2- | TCATGCATCTGGATC | 62. | HES1218 | A7 | | CGA | ABE8e-SaCas9-KKH |
| BE-NNAGAT- | TGGCTGGCCGAGAT | | | | | GAT | |
| Sacas9-A7-23nt | | | | | | | |
| human-ACTA2- | CATGCATCTGGATCT | 63. | HES1216 | A6 | 28 | CGA | ABe8e-SaCas9-KKH |
| BE-NNAGAT- | GGCTGGCCGAGAT | | | | | GAT | |
| SaCas9-A6-22nt | | | | | | | |
| human-ACTA22- | ATGCATCTGGATCTG | 64. | HES1214 | A5 | 27 | CGA | ABB8e-SaCas9-KKH |
| BE-NNAGAT- | GCTGGCCGAGAT | | | | | GAT | |
| SacasS-A5-21nt | | | | | | | |
| human-ACTA2 | TGCATCTGGATCTGG | 65. | | A4 | 26 | CGA | ABE8e-SaCas9-KKH |
| BE-NNAGAT- | CTGGCCGAGAT | | | | | GAT | |
| SaCas9-A4-20nt | | | | | | | |

TABLE B
Target sites across ACTA2-R179H for CRISPR-Cas nucleases

| | | | | target | | | |
|---|---|---|---|---|---|---|---|
| | | | | nucleo- | | | |
| | | SEQ | | tide | target | | |
| | target site with PAM | ID | | posi- | site | | |
| target site name | (R179H bold) | NO: | GRNA ID | tion | length | PAM | use with enzymes: |
| ACTA2-KO-NGG- | GCCCCATGCCATCATGCATCTGG | 66. | HES1234 | 3 | 23 | EGG | WT SpCas9, SpG |
| SpCas9-site1 | | | | | | | |
| ACTA2-KO-NGG- | TGCCATCATGCATCTGGATCTGG | 67. | HES1235 | 9 | 23 | TGG | WT SpCas9, SpG |
| SpCas9-site2 | | | | | | | |
| ACTAZ-KO-NGG- | AGCCAGATCCAGATGCATGATGG | 68. | AES1236 | 7 | 23 | TGG | WT SpCas9, SpG |
| SpCas9-site3 | | | | | | | |
| ACTA2-KO-NGA- | CCCCATGCCATCATGCATCTGGA | 69. | HES1237 | 4 | 23 | GGA | VRQR, VRQR (855R), |
| SpCas9-site4 | | | | | | | SpG, SpRY |
| ACTAZ-KO-NGA- | GCCAGCCAGATCCAGATGCATGA | 70. | | −4 | 23 | TGA | VRQR, VRQR (SSSR), |
| SpCas9-site5 | | | | | | | SpG, SpRY |
| ACTAZ-KO-NGA- | GATCTCGGCCAGCCAGATCCAGA | 71. | | −4 | 24 | AGA | VRQR, VRQR (355R) |
| SpCas9-site6 | T (NGAT > NGAC) | | | | | T | |
| ACTA2-KO-NGC- | TGCCTTGCCCCATGCCATCATGC | | | | 24 | TGC | |
| SpCas9-site7 | A (NGCA > NGCG) | | | | | A | |
| ACTAC-KO-NGC- | GCCATCATGCATCTGGATCTGGC | 73. | | 10 | 23 | GGC | SpG |
| SpCas9-site8 | | | | | | | |
| ACTA2-KO-NGC- | GCCAGATCCAGATGCATGATGGC | 74. | | 3 | 23 | GGC | SpG |
| SpCas9-site9 | | | | | | | |
| ACTAC-KO-NNGGAT- | CTTGCCCCATGCCATCATGCATC | 75, | HES1275 | 2 | 28 | CTG | SaCas9, SaCas9- |
| SaCas9-site10 | TGGAT | | | | | GAT | KKH |
| ACTA2-KO-NNTGAT- | CTCGGCCAGCCAGATCCAGATGC | 76. | HEB1239 | 2 | 28 | CAT | SaCas9-KKR |
| SaCas9-KKA-site11 | ATGAT | | | | | GAT | |
| ACTAS-KO-NNAGAT- | GTGAGATCTCGGCCAGCCAGATC | 77. | HES1240 | −6 | 28 | CCA | SaCas9-KKH |
| SaCas9-KKH-site12 | CAGAT | | | | | GAT | |
| ACTA2-KO-TCCV- | TCCAGATGCATGATGGCATGGGG | 78. | HE81241 | 3 | 24 | TCC | eoAsCas12a, |
| AsCas126-site13 | C | | | | | A | AsCas126 |
| ACTAZ-KO-TCCV- | TCCAGATGCATGATGGCATGGGG | 79. | ABS1242 | 3 | 24 | TCC | LbCasiia |
| LbCas12a-site13 | C | | | | | A | |

The constructs can comprise a promoter, a Kozak sequence, a secretion signal sequence for soluble proteins, a transgene sequence, and a polyadenylation sequence. The woodchuck hepatitis virus posttranscriptional response element (WPRE) can also be used. Optionally, the sequence encoding the transgene is codon optimized, but the human albumin is wild type (not codon optimized).

In some embodiments, the constructs comprise a sequence provided herein, or are at least 80%, 85%, 90%, 95%, 97%, 98%, or 99% identical to a construct sequence set forth herein, optionally omitting any FLAG or other tag sequences, or any plasmid sequences, included in the sequences herein.

### Promoters

The constructs can include a promoter that drives expression of the transgene sequence. In some embodiments, the promoter is a vascular endothelial cell-specific promoter, e.g., VE-cadherin promoter, fms-like tyrosine kinase-1 (FLT-1), intercellular adhesion molecule-2 (ICAM-2), a Claudin 5 (CLDN-5), a von Willebrand factor (vWF) promoter, a TIE2 promoter, or a synthetic EC-specific promoter (see, e.g., Dai et al., J Virol. 2004 June; 78(12): 6209-6221) or SMC-specific promoter as described herein. In some embodiments, the promoter is a pan-cell type promoter, e.g., a “ubiquitous” promoter that drives expression in most cell types, e.g., cytomegalovirus (CMV) promoter (optionally with the CMV enhancer), chicken beta-actin (CBA) promoter, Rous sarcoma virus (RSV) LTR promoter (optionally with the RSV enhancer), SV40 promoter, dihydrofolate reductase promoter, phosphoglycerol kinase promoter, phosphoglycerol kinase (PGK) promoter, EF1alpha promoter, Ubiquitin C (UBC), B-glucuronidase (GUSB), and CMV immediate/early gene enhancer/CBA promoter (CAG); or a steroid promoter or metallothionein promoter. Other promoters may be used, including smooth-muscle specific promoters from HDAC9 or SM22, or promoters from albumin or hepcidin, ENPP1, ENPP2, and ENPP3.

### Kozak Sequence

The constructs typically include a Kozak sequence at the 5′ end of the construct; a consensus Kozak sequence is generally considered as GCCGCCACCATGG (SEQ ID NO:2), where ATG is the start codon.

### Secretory Signal Sequence

A number of secretory signal peptide sequences are known in the art, including human signal sequences, examples of which are shown in Table 2 (Table adapted from novoprolabs.com/support/articles/commonly-used-leader-peptide-sequences-forefficient-secretion-of-a-recombinant-protein-expressed-in-mammalian-cells-201804211337.html).

TABLE 2
Exemplary Human Secretory Signal Peptide Sequences

| Human Signal | | |
|---|---|---|
| sequence | Sequence | SEQ ID NO: |
| Oncostatin M | MGVLLTQRTLLSLVLALLFPSMASM | 3. |
| IgG2 H | MGWSCIILFLVATATGVHS | 4. |
| Secrecon* | MWWRLWWLLLLLLLLWPMVWA | 5. |
| IgKVIII | MDMRVPAQLLGLLLLWLRGARC | 6. |
| CD33 | MPLLLLLPLLWAGALA | 7. |
| tPA | MDAMKRGLCCVLLLCGAVFVSPS | 8. |
| Chymotrypsinogen | MAFLWLLSCWALLGTTFG | 9. |
| trypsinogen-2 | MNLLLILTFVAAAVA | 10. |
| Interleukin 2 (IL-2) | MYRMQLLSCIALSLALVINS | 11. |
| Albumin (HSA) | MKWVTFISLLFSSAYS | 12. |
| Transthyretin | MASHRLLLLCLAGLVFVSEA | 13. |
| ENPP2 | MARRSSFQSCQIISLFTFAVGVNICLG | 14. |
| insulin | MALWMRLLPLLALLALWGPDPAAA | 15. |
| alpha 1-antitrypsin | MPSSVSWGILLLAGLCCLVPVSLA | 16. |

*Barash et al., Biochem Biophys Res Commun. 2002 Jun. 21; 294(4): 835-42.

In some embodiments, another signal sequence that promotes secretion is used, e.g., as described in Table 5 of U.S. Pat. No. 10,993,967, von Heijne, J Mol Biol. 1985 Jul. 5; 184(1):99-105; Kober et al., Biotechnol. Bioeng. 2013; 110: 1164-1173; Tsuchiya et al., Nucleic Acids Research Supplement No. 3 261-262 (2003). In some embodiments, the signal sequence is not an azuricidin signal sequence.

The therapeutic cargo can encode a transgene, e.g., one or more of a transmembrane ENPP1 molecule, a soluble ENPP1 molecule, or both. In some instances, the particle does not comprise a therapeutic cargo encoding a soluble ENPP1 molecule.

### Particles Comprising Cell Targeting Peptides

In some aspects, the disclosure provides “decorated” with peptides on their exterior surface. In order to enhance the targeting of the nanoparticles to specific tissue or cells, the disclosure further conjugated the aforementioned particles with peptides that can target tissue or cell surface receptors. In some embodiments, the LNPs described herein can contain at least one type (e.g., two, three, or four) of targeting peptides covalently-linked to the LNP. Targeting peptides often contain an amino acid sequence that is recognized by a molecule present on the surface of a cell (e.g., a cell type present in a target tissue). For example, a targeting peptide comprising a collagen IV-targeting peptide specifically binds to collagen IV receptors in the extracellular matrix of diseased vasculature. Additional non-limiting targeting peptides, which bind to their respective receptor, and can be covalently-linked to any of the therapeutic nanoparticles described herein include: an interleukin 6 receptor (IL-6R)-targeting peptide, a contiguous sequence of amino acids (e.g., at least 10, 15, or 20) present within IL-6R, a CD63-targeting peptide, a contiguous sequence of amino acids (e.g., at least 10, 15, or 20) present within a contiguous sequence of CD63, a galectin-3 (GAL-3)-targeting peptide, amino acids (e.g., at least 10, 15, or 20) present within Col-4 KLWVLPK-GGG-C(SEQ ID NO: 22), IL6-R C-GGG-LSLITRL (SEQ ID NO: 23), CD63 CRHSQMTVTSRL-GGG (SEQ ID NO: 24), and/or Gal-3 C-GGG-ANTPCGPYTHDCPVKR (SEQ ID NO: 25). Additional examples of targeting peptides are known in the art.

In some embodiments, the targeting peptide is covalently conjugated to the LNP by using a maleimide-terminally modified PEG lipid. Such conjugation includes the reaction of maleimides with thiol groups of the peptides to form thioether bonds. In some embodiments, peptide conjugation can be done in different densities controlled by the percentage of the maleimide-terminally modified PEG lipid in the LNP formulation (see, e.g., Example 5). In some embodiments, the amount of maleimide-terminally modified PEG lipid in the LNP formulation ranges from about 0.15% to about 1.2%. In some embodiments, the number of targeting peptide molecules that can decorate an outer surface of the particles of the disclosure ranges from about 192 targeting peptides per LNP to about 1270 targeting peptides per LNP (e.g., from about 192 to about 420 targeting peptides per LNP, from about 192 to about 609 targeting peptides per LNP, from about 192 to about 901 targeting peptides per LNP, from about 192 to about 1270 targeting peptides per LNP, from about 420 to about 609 targeting peptides per LNP, from about 420 to about 901 targeting peptides per LNP, from about 420 to about 1270 targeting peptides per LNP, from about 609 to about 901 targeting peptides per LNP, from about 609 to about 1270 targeting peptides per LNP, from about 901 to about 1270 targeting peptides per LNP).

In some embodiments, the targeting peptide can be covalently linked to the LNP at its N-terminus or at its C-terminus. In some embodiments, the targeting peptide can be covalently linked to the LNP through an amino acid side chain. Targeting peptides can be covalently-linked to any of the LNPs described herein through a chemical moiety containing a disulfide bond, an amide bond, or a thioether bond. Additional chemical moieties that can be used to covalently link a targeting peptide to a therapeutic nanoparticle are known in the art.

A variety of different methods can be used to covalently link a targeting peptide to a therapeutic nanoparticle. In some embodiments, the LNPs can be activated for attachment with a targeting peptide, for example in non-limiting embodiments, the LNPs can be epoxy-activated, carboxyl-activated, iodoacetyl-activated, aldehyde-terminated, amine-terminated, or thiol-activated. Additional methods for covalently linking a targeting peptide to a therapeutic nanoparticle are known in the art.

In some aspects, the disclosure further conjugated particles comprising from 0.1 to 80% DOTAP with peptides (e.g., collagen IV peptides) that target receptors highly expressed in diseased vasculature extracellular matrix (collagen IV) in order to increase the accumulation and the retention of the nanoparticles in diseased tissues. In some aspects, the disclosure further comprises conjugated particles comprising from 0.1 to 80% DOTAP with peptides that target receptors highly expressed on the surface of vSMCs (e.g., IL-6R, CD63, and/or GAL-3), thereby increasing the uptake into these cells. In some aspects, the disclosure further comprises particles comprising from 0.1 to 80% DOTAP encapsulating a nucleic acid therapeutic cargo encoding a gene (e.g., ENPP1) for rescuing gene expression in a smooth muscle cell. The disclosure demonstrates that a combination of any of these three approaches can potentially increase accumulation and retention at the SMC target site and cell specificity.

In some aspects, the disclosure comprises a particle comprising: a) from 0.1% to 80% of a molecule of formula I

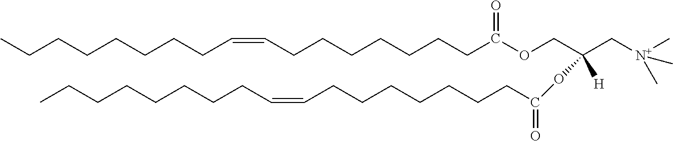 [SMILES: CCCCCCCC/C=C\CCCCCCCC(=O)OC[C@H](C[N+](C)(C)C)OC(=O)CCCCCCC/C=C\CCCCCCCC]

(DOTAP); and a peptide conjugated to a linker molecule in the particle, wherein the peptide has an affinity for a vasculature extracellular matrix molecule. In some aspects, the disclosure comprises a particle comprising: a) from 0.1% to 80% of a molecule of formula I

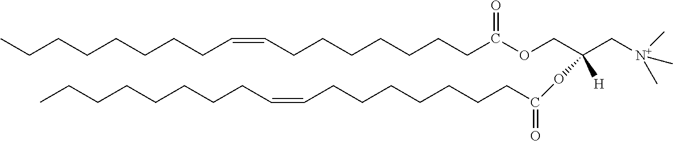 [SMILES: CCCCCCCC/C=C\CCCCCCCC(=O)OC[C@H](C[N+](C)(C)C)OC(=O)CCCCCCC/C=C\CCCCCCCC]

(DOTAP); and a nucleic acid therapeutic cargo encoding a gene for rescuing gene expression in a smooth muscle cell. In many instances, the composition also comprises an amount of an ionizable lipid; an amount of neutral lipid; an amount of cholesterol; and an amount of a PEG-lipid. In many instances, the concentration of DOTAP ranges from 0.1% to 80%, and the molar ratios of the ionizable lipid, the neutral lipid, the cholesterol, and the one or more PEG-lipids are adjusted to a molar ratio of approximately 50/10/38.5/1.5 or about 10/2.1/7.6/1.5. In some embodiments, the amount of the ionizable lipid ranges from about 45% to about 52% or about 5% to about 15%. In some embodiments, the amount of the neutral lipid ranges from about 1% to about 3% or 9% to about 11%. In some embodiments, the amount of cholesterol ranges from about 5% to about 9% or about 34% to about 40%. In some embodiments, the amount of the one or more PEG-lipids range from about 0.1% to about 2%.

In some aspects, the linker molecule is a molecule that is used to covalently link the peptide to the LNP. In some embodiments, the linker molecule is a maleimide group at a PEG lipid in the particle as discussed supra. The peptide can be a collagen IV (Col-IV) peptide, an IL-6R peptide, a CD63, a GAL-3, and/or a functional fragment thereof sufficient for increasing an accumulation and the retention of the nanoparticles in target tissues. The neutral lipid can be a phosphatidylcholine lipid or a phosphatidylethanolamine lipid, such as the ones selected from the group consisting of DOPE, DOPC, DSPC, DPPC, POPC, and SOPC. The ionizable lipid can be selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DM A, DLin-K-C4-DMA, DLen-C2K-DMA, 7-DLen-C2K-DMA, or DLin-MP-DMA.

Such particles can encapsulate any one of the aforementioned nucleic acid therapeutic cargo(s). In some embodiments, the therapeutic cargo is an mRNA molecule encoding a gene for rescuing gene expression in the smooth muscle cell. In some embodiments, the therapeutic cargo is a plasmid encoding a gene for rescuing gene expression in the smooth muscle cell. In some embodiments, the therapeutic cargo is a nucleic acid molecule encoding an ENPP1 therapeutic cargo. In some embodiments, the ENPP1 therapeutic cargo encodes a transmembrane ENPP1 molecule. In some embodiments, the ENPP1 therapeutic cargo encodes a soluble ENPP1 molecule. In some embodiments, the transmembrane ENPP1 molecule is SEQ ID NO: 1.

In some aspects, the disclosure comprises a therapeutic formulation comprising: from 0.1% to 80% of a molecule of formula I

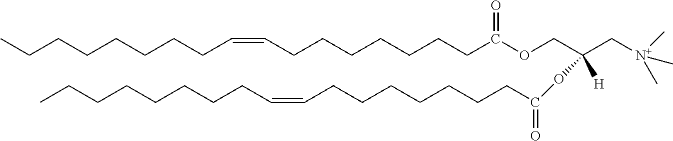 [SMILES: CCCCCCCC/C=C\CCCCCCCC(=O)OC[C@H](C[N+](C)(C)C)OC(=O)CCCCCCC/C=C\CCCCCCCC]

(DOTAP); an amount of an ionizable lipid; an amount of neutral lipid; an amount of cholesterol; an amount of a PEG-lipid; comprising amounts of the ionizable lipid, the neutral lipid, the cholesterol, and the one or more PEG-lipids at a molar ratio of 50/10/38.5/1.5; and a nucleic acid construct encoding an ectonucleotide pyrophosphatase/phosphodiesterase 1 (ENPP1) therapeutic cargo. The ENPP1 therapeutic cargo can be a transmembrane ENPP1 molecule or a soluble ENPP1 molecule. In some instances, the particle does not comprise a cargo encoding a soluble ENPP1 molecule. In some instances, the disclosure provides a therapeutic formulation comprising: about 10% of DOTAP; about 46% of an MC3 ionizable lipid; about 9.8% of a DOPE neutral lipid; about 35.3% of cholesterol; about 1.4% of one or more PEG-lipids; and a nucleic acid construct encoding an ectonucleotide pyrophosphatase/phosphodiesterase 1 (ENPP1) therapeutic cargo. In some instances, the disclosure provides a therapeutic formulation comprising: about 80% of DOTAP; about 10% of an MC3 ionizable lipid; about 2.1% of a DOPE neutral lipid; about 7.6% of cholesterol; about 1.5% of one or more PEG-lipids; and a nucleic acid construct encoding an ectonucleotide pyrophosphatase/phosphodiesterase 1 (ENPP1) therapeutic cargo.

In some aspects, the disclosure comprises a therapeutic formulation comprising: a) from 0.1% to 80% of a molecule of formula I

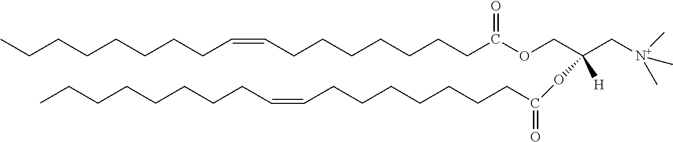 [SMILES: CCCCCCCC/C=C\CCCCCCCC(=O)OC[C@H](C[N+](C)(C)C)OC(=O)CCCCCCC/C=C\CCCCCCCC]

(DOTAP); and an amount of an ionizable lipid; an amount of neutral lipid; an amount of cholesterol; an amount of one or more PEG-lipids; comprising amounts of the ionizable lipid, the neutral lipid, the cholesterol, and the one or more PEG-lipids at a molar ratio of 50/10/38.5/1.5; and a nucleic acid construct encoding an smooth muscle alpha (α)-2 actin (ACTA2) therapeutic cargo.

### Pharmaceutical Compositions

Also provided herein are pharmaceutical compositions that contain a therapeutic LNP as described herein. Two or more (e.g., two, three, or four) of any of the types of therapeutic LNPs described herein can be present in a pharmaceutical composition in any combination. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerin, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfite, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the LNP compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the therapeutic nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid.

Compositions containing one or more of any of the therapeutic LNPs described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Dosage, toxicity and therapeutic efficacy of the therapeutic constructs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Constructs that exhibit high therapeutic indices are preferred. While constructs that exhibit toxic side effects may be used, care should be taken to design a delivery system that targets such constructs to the site of affected tissue in order to minimize potential damage to unaffected cells and, thereby, reduce side effects.

Data obtained from cell culture assays and animal studies can be used in formulating a range of dosage for use in humans. The dosage of such constructs lies preferably within a range of circulating concentrations that include the ED50 with little or no toxicity. The dosage may vary within this range depending upon the dosage form employed and the route of administration utilized. For any construct used in a method described herein, a therapeutically effective dose can be estimated initially from animal models or based on other constructs. Such information can be used to more accurately determine useful doses in humans.

An “effective amount” is an amount sufficient to effect beneficial or desired results. For example, a therapeutic amount is one that achieves a desired therapeutic effect. This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The LNP compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic LNP compositions described herein can include a single treatment or a series of treatments.

The pharmaceutical compositions can be included in a container, pack, or dispenser together with instructions for administration.

### Methods of Delivering LNPs and Therapeutic Cargos to Vascular SMCs

Provided herein are methods of delivering any of the LNPs of the disclosure, including or excluding any of the therapeutic cargos of the disclosure, to a subject who has a condition associated with vascular calcification. The methods of delivery comprise the administration of any of the LNPs and/or LNP compositions described herein to a subject. In some aspects, the disclosure provides a method of delivering a nucleic acid therapeutic cargo to a smooth muscle cell, the method comprising administering to or contacting a smooth muscle cell with any one of the particles described herein. In some aspects, the therapeutic cargo may lower a blood pressure of the subject (e.g., reduced levels of calcification in a subject may be inferred from lower blood pressures).

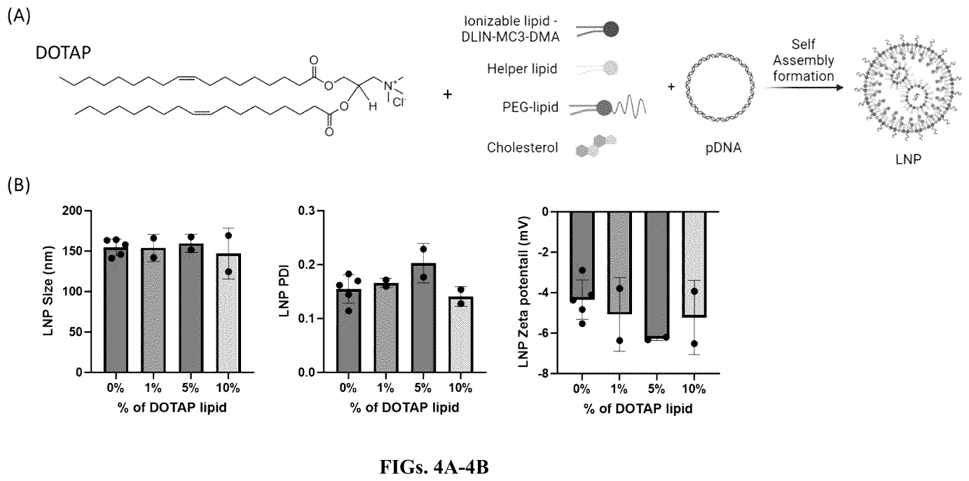

Diseases that can be treated using the methods and compositions described herein include hypertension, GACI, pseudoxanthoma elasticum (PXE), calciphylaxis, and cardiovascular disease including diabetic vascular calcification, ESRD-associated vascular disease, calcific aortic valve disease (CAVD), coronary atherosclerosis, peripheral vascular disease, and cerebral atherosclerosis. In general, the methods include administering a therapeutically effective amount of LNPs encapsulating a nucleic acid cargo (e.g., an ENPP1 gene construct, optionally including a CBA, CMV, CAG, or other promoter). The LNPs are preferably administered intravenously, but can also be administered by other routes such as subcutaneous injection as described supra. Exemplary LNP compositions are described herein (e.g., see FIGS. 4A-B, 7A-B, 11, 13, and 15A). Exemplary nucleic acid cargos are described herein. Non-limiting examples of nucleic acid cargos include ENPP1 and those described in, for example, Table 1.

Conditions treatable using the present methods and compositions include the following:

Generalized arterial calcification of infancy (GACI): GACI is characterized by widespread arterial calcification and/or stenoses of large and medium-sized vessels resulting in a range of clinical manifestations including myocardial infarction, respiratory distress, hypertension, cardiomegaly, and stroke. GACI is estimated to affect one in 200,000 pregnancies. Mortality is particularly high in early infancy; approximately 55% of patients die within the first 6 months of life despite intensive care and supportive measures. After 6 months of life, the mortality rate is markedly reduced and patients tend to survive, though many still have sequalae from their initial hypoxic insults, and a majority eventually develop hearing loss and hypophosphatemic rickets. GACI typically results from biallelic loss-of-function mutations in ENPP1, which encodes an ectonucleotide pyrophosphatase/phosphodiesterase that converts ATP into AMP and pyrophosphate (PPi), a potent inhibitor of calcification. Loss of ENPP1 activity results in decreased quantity of PPi both locally and systemically, and GACI patients have low plasma and urinary PPi concentrations. A treatment as described herein can result in reduced arterial calcification and/or stenoses of large and medium-sized vessels, and increased plasma and/or urinary PPi concentrations (approaching, near, or within normal levels; generally 0.1 ug/ml in Asj2 mice).

Pseudoxanthoma elasticum (PXE): PXE is caused by defects in the presumptive ATP-dependent exporter ABCC6, disrupts extracellular ATP metabolism resulting in calcification of elastic fibers in the skin, eyes, and arterial wall. Furthermore, mutations in ENPP1 have been described in patients with PXE. Null mice (Abcc6−/−) recapitulate the genetic, histopathologic and ultrastructural features of PXE, and they demonstrate early and progressive mineralization of vibrissae dermal sheath, which serves as a biomarker of the overall mineralization process. A treatment as described herein can result in reduced calcification of elastic fibers in the skin, eyes, and arterial wall, and/or reduced mineralization of vibrissae dermal sheath.

Calciphylaxis: Calciphylaxis is a rare, life-threatening disease of rapidly progressive vascular calcification characterized by microvascular occlusion in the dermis and subcutaneous tissue. Patients with calciphylaxis have limited survival of typically less than one year. They also have significant morbidity from cutaneous pain and soft tissue infections, often requiring surgical debridement and amputation. Traditionally observed in patients with end-stage kidney disease (ESKD; e.g., ˜1% of hemodialysis patients have calciphylaxis), calciphylaxis is also associated with diabetes, hyperphosphatemia, and warfarin use. However, the molecular mechanisms of calciphylaxis are incompletely understood, which has precluded the development of approved therapies. Recent investigations have demonstrated that the process of small vessel arteriolar calcification in calciphylaxis exhibits similarities to that of large artery calcification. Coronary and aortic calcification are characterized by the phenotypic switch of vascular smooth muscle cells (VSMCs) from contractile to osteogenic cells, which is induced by Runt-related transcription factor 2 (Runx2) and vitamin K-dependent modulation of matrix Gla protein (MGP) and the bone morphogenetic protein (BMP) signaling pathway. Similarly, in calciphylaxis, there is increased expression of osteogenic markers as well as proteins associated with altered remodeling of the extracellular matrix. Vitamin K deficiency-mediated reduction in carboxylated MGP (a known inhibitor of BMP signaling) is associated with increased risk of calciphylaxis in patients on hemodialysis. Patients with calciphylaxis have reduced pyrophosphate levels compared to matched ESKD patients (unpublished data), implicating ENPP1 in the pathogenesis of calciphylaxis. A treatment as described herein can result in reduced vascular calcification.

Cardiovascular disease: Vascular calcification plays an important role in human arterial disease (e.g., in atherosclerosis and diabetes). Cardiovascular disease is the leading cause of morbidity and mortality in the world. In the United States alone, cardiovascular disease accounts for over 780,000 deaths annually. Vascular calcification is a hallmark of atherosclerotic disease and serves as strong predictor and risk factor for cardiovascular events. Two primary types of vascular calcification have been reported in adults: intimal calcification, associated with atherosclerosis, and medial calcification, associated with chronic kidney disease and diabetes. Intimal calcification occurs in the setting of lipid accumulation and macrophage infiltration into the vessel wall. Medial wall calcification localizes to elastin fibers or smooth muscle cells and is not associated with lipid deposition or macrophage infiltration. Intimal calcification of the atherosclerotic vessel wall is thought to contribute to plaque destabilization and predicts increased risk in cardiovascular disease. Calcification of the medial vessel layer also predicts cardiovascular events and is associated with increased wall stress, pulse pressure, and risk of rupture in aortic aneurysms. Vascular calcification is a tightly regulated process and overlap exists in the molecular underpinnings of atherosclerotic intimal calcification and medial calcification. An increased understanding of the mechanisms that lead to arterial calcification will have important clinical implications for a broad range of cardiovascular diseases and will facilitate identification of novel therapeutic targets. More recently, in work that is currently under review, we have found common variants (single nucleotide polymorphisms, SNPs) in the ENPP1 locus that are associated with coronary artery calcification at a genomewide level of significance. These SNPs that are associated with increased risk of coronary calcification are also associated with lower expression of ENPP1. These findings implicate relative ENPP1 deficiency in the development of calcification and cardiovascular disease in the general population. A treatment as described herein can result in reduced blood pressure, reduced vascular calcification, and reduced risk of cardiovascular disease.

Calcific Aortic Valve Disease (CAVD): CAVD is the most prevalent cardiac valvular disease among elderly individuals and the prevalence of CAVD is increasing with ˜5% of all individuals above the age of 75 affected. A 3.5-fold increased annual incidence now compared to 30 years ago has been reported. 41 CAVD progresses from mild calcification of the valve leaflets to severe calcification and narrowing of the aortic valve orifice, resulting in an obstruction to forward blood flow from the left ventricle and left ventricular hypertrophy. No medical treatment exists for CAVD and treatments are surgical including surgical aortic valve replacement (SAVR) or transcatheter aortic valve implantation (TAVI). An ex vivo study of aortic valve leads demonstrated an important role for endogenous pyrophosphate in the inhibition of valvular calcification. Furthermore, a combined proteomic-metabolomic analysis of CAVD vs control aortic valve tissue identified ENPP1 as a hub protein in the metabolite-protein-pathway network. This evidence points towards ENPP1 as a potential therapeutic in aortic valve calcification. Multiple animal models for CAVD exist that overlap with models of atherosclerosis and vascular calcification. A treatment as described herein can result in reduced aortic valve calcification.

### Terminology

The singular forms “a,” “an,” and “the” include plural reference unless the context clearly dictates otherwise. Thus, for example, a reference to: “a lipid” includes a plurality of such lipids, so that a lipid “X” (e.g., cholesterol, ionizable lipid, permanently cationic lipid, PEG lipid, and/or neutral lipid) includes a plurality of one or more such lipid molecules. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. Similarly, fractional numbers encompassed within the range are also contemplated by the disclosure. A recited range (e.g., weight percentages or lipid groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

As will be understood by the skilled artisan, all numbers, including those expressing quantities of ingredients, properties such as molecular weight, reaction conditions, and so forth, are approximations and are understood as being optionally modified in all instances by the term “about.” The terms “about” and “approximately” are used interchangeably. Both terms refer to a variation of ±10% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range up to +10%. Unless indicated otherwise herein, the terms “about” and “approximately” are intended to include values, e.g., molar percentages and/or weight percentages, proximate to the recited range that are equivalent in terms of the functionality of the individual ingredient, composition, or embodiment. The terms “about” and “approximately” can also modify the endpoints of a recited range as discussed above in this paragraph.

The “subject(s)” referred throughout the specifications include, but are not limited to, humans and non-human mammals. In some embodiments, the subject being treated is a human. In some embodiments, the subject being treated is a laboratory animal (e.g., used in research), such as a rodent, a rabbit, a sheep, or a primate.

In some embodiments, the subject being treated in accordance with the methods described herein has a deficiency in expression of a gene product encoded by the therapeutic cargo. In some embodiments, the subject being treated in accordance with the methods described herein has a mutation in or a deletion of the gene that the transgene is used to replace. In some embodiments, the subject being treated in accordance with the methods described herein has a loss-of-function mutation in the gene or in another genomic location that affects gene expression of the gene.

### EXAMPLES

The invention is further described in the following examples, which do not limit the scope of the invention described in the claims.

### Example 1—Materials and Methods

The following materials and methods were used in the examples herein. However, various LNP formulations were prepared and utilized in the examples below. The specific LNP formulations are specified in each example and/or corresponding figure(s).

### Methods

### Lipid Nanoparticles Formulation and Peptide Conjugation

LNPs were formulated by pipette mixing a stock of organic phase composed of a lipid mixture with an aqueous phase composed of mRNA dissolved in 10 mM sodium acetate buffer, pH 5.2 (Sigma, 567422) at a volume ratio of 1:3 organic to aqueous. DLin-MC3-DMA (MC3) (Eschelone Bioscience, N-1282) Ionizable lipid, 1,2-dioleoyl-sn-glycero-3-phosphoethanolamine (DOPE, Avanti Polar Lipids, 850725P), cholesterol (Sigma, C8667) and 1,2-dimyristoyl-rac-glycero-3-methoxypolyethylene glycol-2000 (DMG-PEG 2000, Avanti Polar Lipids, 880151P) were dissolved in ethanol and mixed at predetermined molar ratios (50:10.5:38:1.5) and a 40:1 total lipids to mRNA weight ratio. The assembled LNPs were dialyzed against PBS (pH 7.4) in dialysis tubes (3500 MW cutoff, Sigma, PURD35050) to remove the ethanol. In formulations where 1,2-dioleoyl-3-trimethylammonium-propane (chloride salt) (DOTAP) was incorporated, the percentage of DOTAP was considered as part of the total 100% while the ratios between the remaining lipids was kept (e.g., 50:10.5:38:1.5 or 10:2.1:7.6:1.5:78.8). Peptide-conjugated LNPs are composed of, for example, 10% DOTAP and DSPE-PEG 2000 (1,2-distearoyl-sn-glycero-3-phosphoethanolamine-N-[maleimide(polyethylene glycol)-2000](ammonium salt) in varying percentages (0.15%/0.3%/0.6%/0.9%/1.2%) as replacement for DMG-PEG 2000 (total lipid PEG ratio did not exceed 1.5%).

Other peptide-conjugated LNPs used in this study are composed of, for example, 78.8% DOTAP, DSPE-PEG-mal (1,2-distearoyl-sn-glycero-3-phosphoethanolamine-N-[maleimide(polyethylene glycol)-2000](ammonium salt) in varying percentages (e.g., 0.3% and 0.6%), DMG-PEG in varying percentages (e.g., 0.3%, 0.9%, and 1.2%), 7.6% cholesterol, 2.1% DOPE, and 10% MC-3, as described in Table 6 below.

In this study, the peptides under investigation were designed with dual objectives: firstly, to target the extracellular matrix of vascular smooth muscle cells (VSMCs) with a peptide targeting collagen 4 (Col-4; KLWVLPKGGGC, SEQ ID NO: 22), thereby enhancing the retention of LNPs around these cells; and secondly, to target surface receptors expressed on VSMCs to facilitate the internalization of LNPs. The sequences of the peptides targeting surface receptors were IL6R: CGGGLSLITRL, SEQ ID NO: 23; Gal-3: CGGGANTPCGPYTHDCPVKR, SEQ ID NO: 25; and CD63: CRHSQMTVTSRL, SEQ ID NO: 24. For conjugating peptides to LNPs, the peptides were initially treated with tris(2-carboxyethyl)phosphine (TCEP) reducing agent (Thermo Scientific, 77712). Subsequently, the free peptides were incubated with Mal-functionalized LNPs for 1 hour at pH 7.4, with a peptide to Mal molar ratio of 2:1. The final product of peptide-conjugated LNPs was dialyzed against phosphate buffered saline (PBS) and concentrated using Amicon® tubes. To assess the efficiency of conjugation, a maleimide fluorometric detection kit (Sigma, MAK167) was utilized following the protocol of the manufacturer.

### LNPs Characterization

The size (diameter) and surface charge (zeta potential) of the LNPs were determined via dynamic light scattering (DLS) using the Zetasizer® Nano ZS (633 nm, Malvern Instruments), with light collection at a scattering angle of 173°. Nucleic acid encapsulation efficiency was assessed employing the modified Quant-it™ RiboGreen® RNA assay (Invitrogen™). Initially, a nucleic acid standard curve was prepared in TE (Tris-EDTA) buffer. LNPs were subsequently diluted either in TE buffer or TE-Triton (TE supplemented with 2% Triton-X100) to match the nucleic acid concentration within the standard curve. Duplicates of LNPs and standard curve samples (100 μL each) were loaded into a black 96-well plate, followed by the addition of 100 μL of RiboGreen® reagent (diluted 1:200 in TE buffer). Fluorescence intensity originating from unencapsulated nucleic acid (I_{free}) was measured using a plate reader (Excitation: 485 nm, Emission: 528 nm, Infinite® M plex, TECAN®). Triton-treated LNP samples represented released nucleic acid from disrupted LNPs, while intact intensity of LNPs indicated unencapsulated nucleic acid. Encapsulation efficiency was computed as % EE=100×(1−(I_{intact}/I_{disrupted})).

In Vitro LNPs Transfection Studies Encapsulating mRNA Expressing GFP

Mouse smooth muscle cells, MOVAS, were cultured in DMEM media (Thermo 11965118) supplemented with 10% FBS and 5% Pen-Strep. 15,000 cells were seeded in a 48-well plate 24 h prior treatment. The cells were treated with 100 ng of mRNA per well in complete media, encapsulated in LNPs with varying DOTAP percentages. For peptide studies, cells were treated with either 10% DOTAP LNPs non-Mal-functionalized (named DOTAP) or Mal-functionalized with either non-conjugated or conjugated peptide. 24 h post treatment cells were harvested and fixed for determination of GFP expression via flow cytometry acquisition (BD LSRFortessa™ flow cytometer, BD Biosciences) and data analysis by FlowJo™ software. Cell viability was confirmed to validate flow results. Cell viability was done before flow using the PrestoBlue™ viability assay (Thermo Scientific, A13262) according to the protocol of the manufacturer.

### In Vivo Gene-Editing Study in Marfan/tdTom Mouse Model

Marfan mice (B6.129-Fbn^{1tm1Hcd}/J)(MFS) were obtained from Jackson labs. Male Marfan mice were bred to homozygous (B6.Cg-Gt(ROSA)26Sor^{tm14(cAG-tdTomato)Hze}/J)(TdTom:TdTom) female mice to produce MFS:TdTom and TdTom offspring of both sexes for LNP injections. LNPs encapsulating Cre-mRNA (TriLink®, L-7211) were injected retro-orbitally into p3 mice at a dose of 1 mg/kg. Organ tdTom expression was determined 7 days post injection in which mice were perfused with PBS and organs were dissected for imaging. tdTom expression was determined by Sapphire™ Biomolecular fluorescent imager (Azure Biosystems) using Ex./Em. 550/580 nm and 200 μm organ scanning resolution. Organ fluorescence intensity was analyzed by Fiji software and intensity was normalized to the untreated (PBS injected) group within each organ comparison.

### Example 2: Particle Screening for Delivery of Constructs to Liver Hepatocytes to Produce a Soluble Protein

The instant example describes screening of LNP composition(s) for efficient encapsulation of plasmid constructs. Screening was based on alteration of the cholesterol and DOPE lipid ratio while fixing the ionizable and DMG-PEG lipid amounts. Screening based on cholesterol and DOPE content was classified by high, medium, or low cholesterol amount as outlined in Table 3.

TABLE 3

| | Cholesterol Content | | |
|---|---|---|---|
| | High | Medium | Low |
| MC-3 | 50% | 50% | 50% |
| DOPE | 10% | 30% | 40% |
| PEG-DMG | 1.5% | 1.5% | 1.5% |
| Cholesterol | 38.5% | 18.5% | 8.5% |

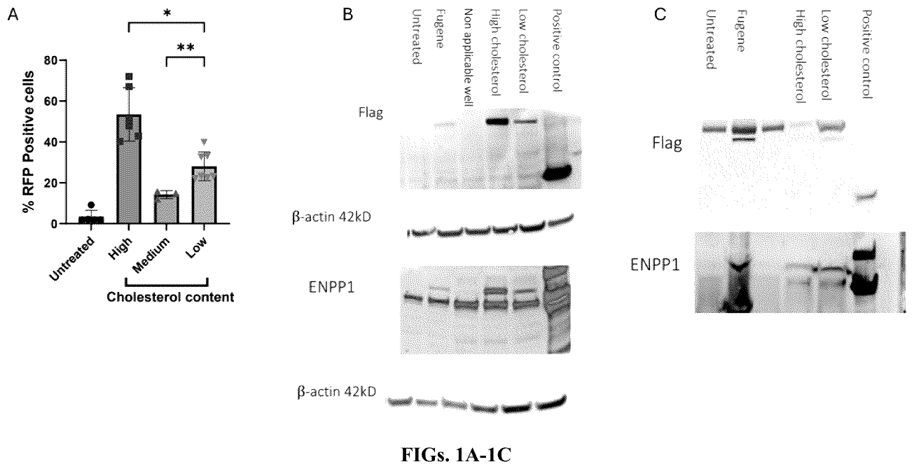

The particles were screened for the delivery in liver hepatocytes (HepG2) cell line based on formulation composition varying in cholesterol and DOPE content illustrated in Table 3. It was found that a composition with high cholesterol was the most efficient in delivering a reporter plasmid expressing RFP, as well as delivering a plasmid encoding a soluble ENPP1 protein in liver hepatocyte cells in vitro (FIGS. 1A-C). FIG. 1A is a chart illustrating results of a flow cytometry experiment quantitating the percentage of RFP positive cells in the liver hepatocytes (HepG2) cell line. FIGS. 1B-C illustrate the detection of plasmid constructs expressing soluble ENPP1 and detection at cell lysates (FIG. 1B) and detection in cell media of ENPP1 protein expressed from constructs expressing the soluble protein.

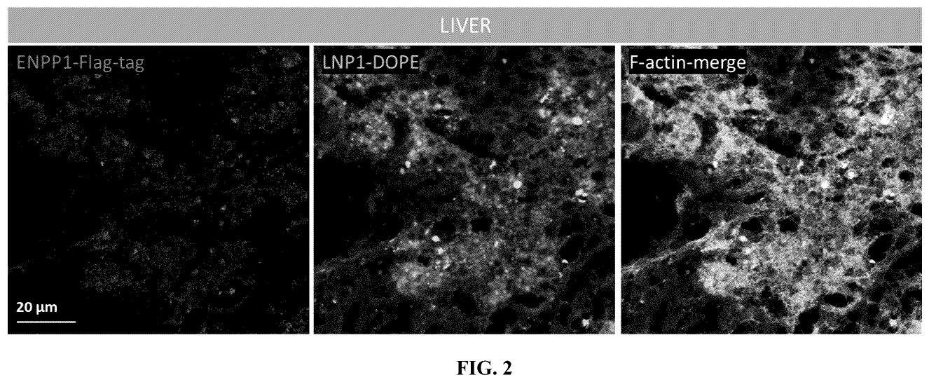

The preferential delivery of an ENPP1 soluble construct in a mouse model for the generalized arterial calcification of infancy (GACI) disease (Asj mouse model of ENPP1 deficiency (FIG. 2)) was further demonstrated. This experiment demonstrated efficient soluble ENPP1 expression in the liver of Asj mouse models among other organs 6 days post injection. FIG. 2 are histological images of liver of Asj mice injected with soluble ENPP1 plasmid (0.3 mg/kg) at day 3 (P3) express high levels of the enzyme in the liver, 6 days post injection, demonstrating in vivo expression of a construct delivered with the particles described in Table 3 to the liver.

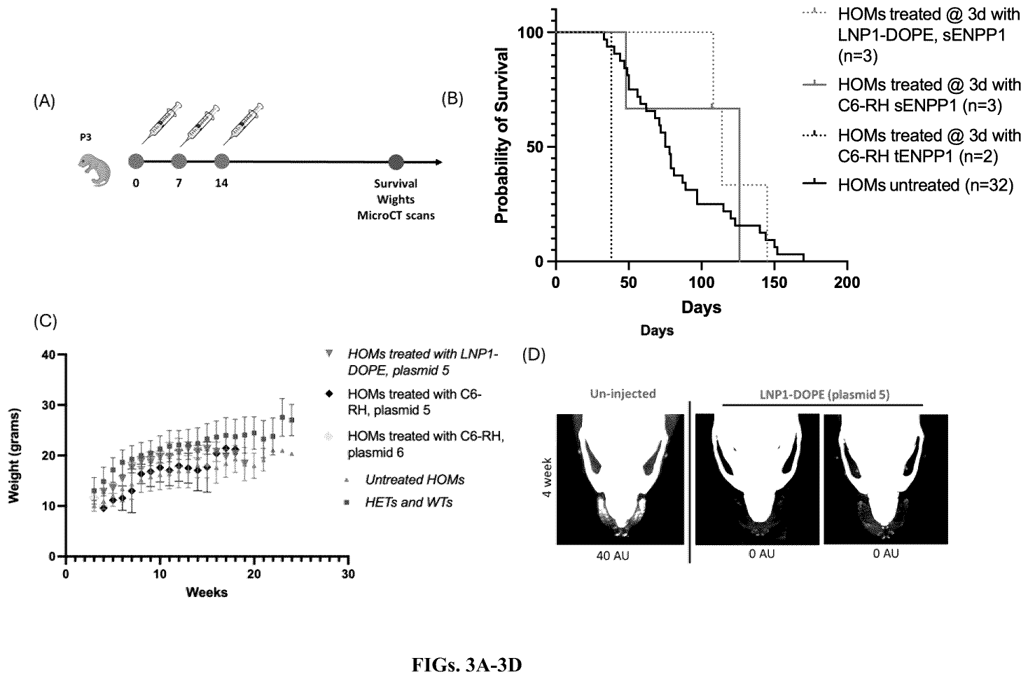

The outcome of treatment of soluble srENPP1 constructs delivered with LNPs with high cholesterol content (see Table 3) was further evaluated. The particles and compositions used in the delivery provided measurable survival benefits extending the life expectancy of treated animals from 80 days (untreated animals) to 114 days (treated animals) (n=3). In addition, microCT scans showed reduced calcification in the vibrissae of treated diseased animals in comparison with untreated group (FIGS. 3A-D). FIGS. 3A-D depict results of LNPs delivery efficacy studies using plasmids expressing soluble srENPP1. FIG. 3A is an illustration presenting the injection regimen. Briefly P3 mice were injected at day 0, day 7, and day 14. FIG. 3B are survival curves of animals. FIG. 3C is a chart illustrating animal body weight. FIG. 3D are MicroCT scans to detect early development of calcification in treated (LNPs encapsulating soluble ENPP1, 0.3 mg/kg) and untreated animals.

The experiment demonstrated (1) the efficacy of the particles in delivering the plasmid construct encoding the soluble ENPP1 construct in vivo; and (2) the effectiveness of the treatment as measured by increased survival, reversal of calcification, animal body weight.

### Example 3: Particle Screening for Delivery of Therapeutic Cargos to Smooth Muscle Cells (SMCs)

Having established the therapeutic effective of a plasmid construct delivered with high cholesterol particles, particles that displayed select tropism to smooth muscle cells were screened in order to further improve delivery of the treatment.

To target nanoparticles to SMCs, a particle formulation based on the addition of a fifth component, DOTAP, a permanently cationic lipid (FIGS. 4A-B), was developed. It was reasoned that introducing DOTAP to the LNP composition at low percentage affects the chemical nature of the nanoparticles and increases the interactions with SMCs which is followed by cell internalization and construct expression. Importantly, a significant effect of the DOTAP with regards to particle size, size distribution, and zeta potential of the LNPs was not observed at the range that was screened (up to 10% DOTAP, FIGS. 4A-B).

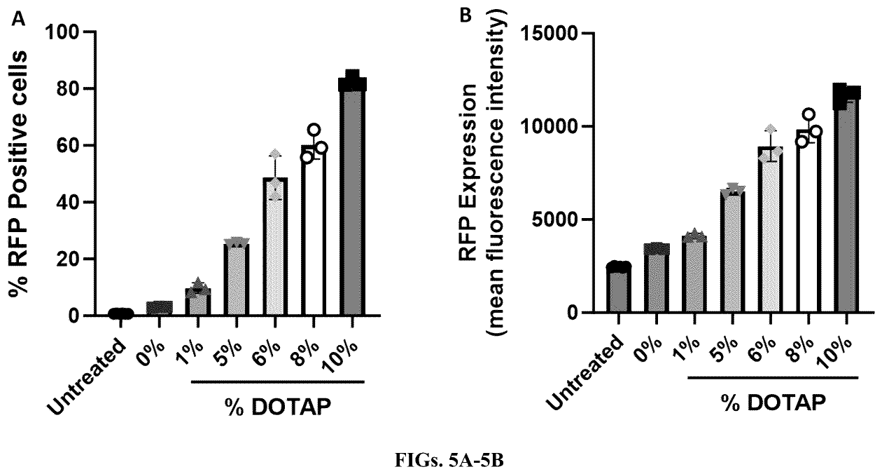

Second, the transfection efficiency and expression of a model plasmid expressing RFP in mouse SMCs line (MOVAS) was evaluated. It was found that the presence of the permanently positive cationic lipid DOTAP, as well as the concentration of DOTAP, to be highly crucial for successful transfection of MOVAS cells. In contrast to the compositions incorporating DOTAP, the conventional four-component compositions of the art consisting only of ionizable lipids failed to demonstrate any expression. FIGS. 5A-B are graphs depicting the results of the introduction of DOTAP lipid into the four-component formulation for screening its efficiency in delivering a plasmid cargo to MOVAS cells. As depicted in FIGS. 5A and 5B, the addition of as little as 1% DOTAP into the formulation drove an increase in cells expressing RFP (FIG. 5A for the percentage of positive cells; FIG. 5B for the mean fluorescence intensity). In this experiment, MOVAS cells were incubated for 48 h with the reporter plasmid (2 μg/48 well plate) encoding RFP. Cellular expression was identified and measured using flow cytometry.

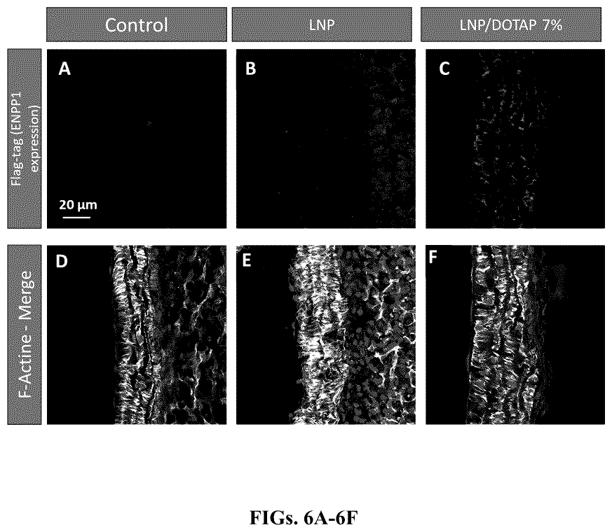

In addition to in vitro data, the role of DOTAP in the LNP formulation in the context of delivery of a plasmid nucleic acid encoding a therapeutic cargo to SMCs in vivo was confirmed. Using the Asj mouse model of ENPP1 deficiency for the generalized arterial calcification of infancy (GACI) disease, successful delivery of plasmid DNA encoding the transmembrane rENPP1 expression at SMCs at the aorta (indicated by the SMC labeled by F-actin) was demonstrated (FIGS. 6A-F). Further, it was demonstrated that the conventional LNP formulations with four components do not demonstrate SMC expression in vivo (FIGS. 6B, 6E). The expression of the control LNPs of the art appear to be mainly localized at the adventitia. It was further confirmed that the described DOTAP formulations efficiently expressed a construct encoding a therapeutic gene, namely ENPP1 (FIGS. 6C, 6F). In this experiment, the Asj mouse model at P3 was injected systemically with PBS (control), LNPs (conventional four-component formulation of the art), and DOTAP LNPs formulation (7% DOTAP) at 0.3 mg/kg plasmid dose. Histological images of the aorta show that ENPP1 expression (Flag-tag) colocalized with SMCs marker expression (F-actin).

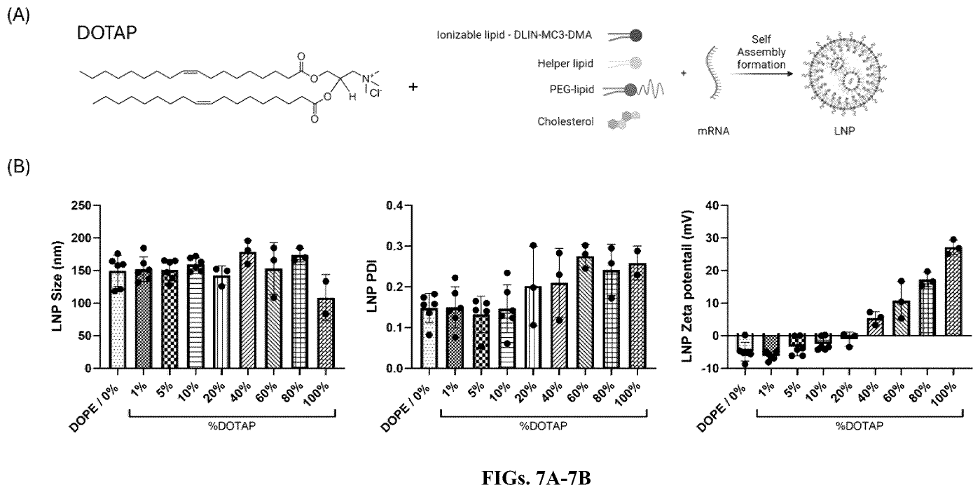

Further, it was observed that the addition of a high percent of DOTAP (e.g. 50%) into the formulation increases tropism to the lung. In contrast, it was observed that the addition of DOTAP at relatively low percentages (<10%) enhanced uptake to SMCs without driving tropism to the lung (as our target in some cases is the liver). It was observed that the addition of DOTAP did not significantly affect the size of the formulated LNPs. However, the size distribution (PDI) increased with higher percentages of DOTAP, possibly due to the decreased proportions of the remaining lipids, which play a crucial structural role in particle formation. Furthermore, the zeta potential increased with higher DOTAP percentages, attributed to the substantial contribution of DOTAP quaternary amine head groups to the total surface charge of the nanoparticles. Despite varying percentages of DOTAP, encapsulation efficiency remained consistently above 90%, facilitating meaningful comparison between different formulations (FIGS. 7A-B).

The present disclosure provides the first demonstrated ability of this technology to deliver the claimed particles to smooth muscle cells, particularly to vascular smooth muscle cells (vSMCs).

### Example 4: Delivery of Gene Therapy Cargos to Particles Targeting Smooth Muscle Cells (SMCs)

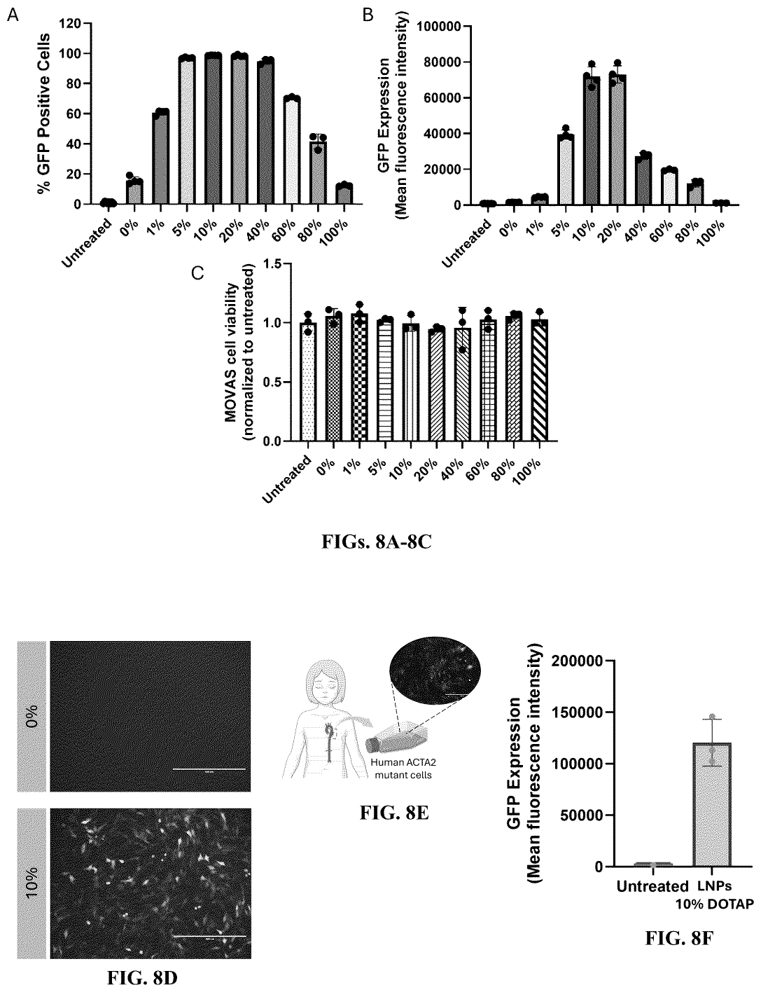

Further validation of the particles comprising the permanently cationic DOTAP component was sought by testing the delivery of different therapeutic cargos. In this experiment, an mRNA therapeutic cargo (as opposed to the plasmid therapeutic cargo of the aforementioned example) was first packaged into the DOTAP particles described above. As a threshold matter, it was determined that the nature of the nucleic acid therapeutic cargo (e.g., plasmid versus mRNA) appeared to have a minimum, insignificant effect on the size, size distribution, and zeta potential of the DOTAP LNPs encapsulating mRNA at the DOTAP ranges screened (up to 10%, FIGS. 7A-B). It was found that the DOTAP LNPs formulation increased the transfection of mRNA in VSMCs in vitro using the delivery of mRNA expressing GFP (FIGS. 8A-B). Moreover, in vitro studies using the MOVAS cell line demonstrated that increasing the content of DOTAP LNPs enhanced mRNA transfection efficiency in VSMCs, reaching an optimum at 10-20% DOTAP. Beyond this range, further increases in DOTAP content led to diminished mRNA delivery and reduced GFP expression (FIGS. 8A-B). Cell viability was not impacted under the different experimental conditions, thus ruling out reduced expression due to cell death (FIG. 8C).

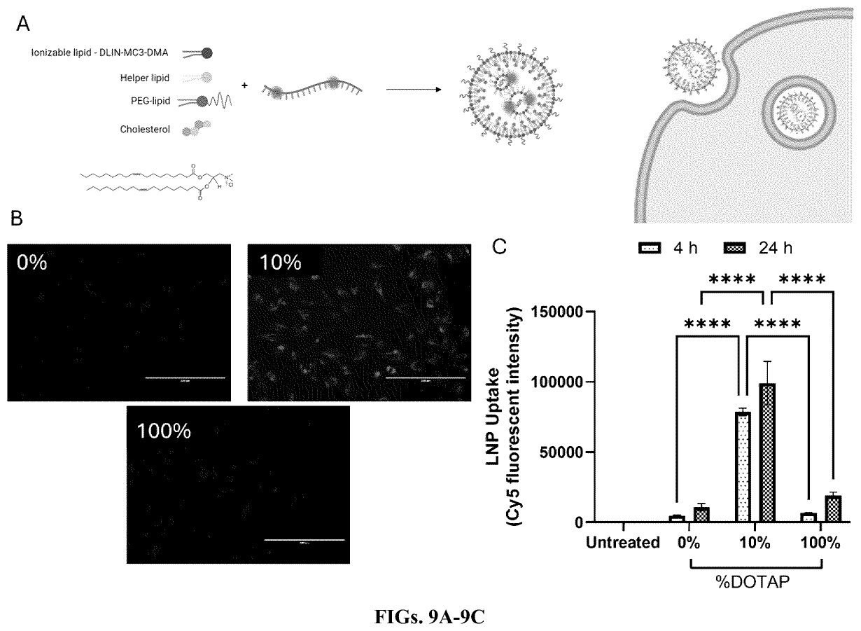

To investigate the underlying cause for the observed optimal mRNA delivery, cellular uptake studies were conducted based on the hypothesis that DOTAP may enhance LNP uptake owing to its positively charged head groups. MOVAS cells were incubated with LNPs or DOTAP LNPs encapsulating Cy5-labeled mRNA for 4 and 24 hours (FIG. 9A). At each time point, cells were visualized using a fluorescent microscope to qualitatively assess mRNA internalization and were subsequently harvested and analyzed by flow cytometry for quantitative evaluation.

The results indicated that the efficient mRNA delivery facilitated by 10% DOTAP LNPs was attributed to enhanced cellular uptake, evidenced by an increased Cy5 intracellular signal (FIG. 9B). Specifically, as shown in FIG. 9C, a 20-fold increase in fluorescent intensity was observed with 10% DOTAP LNPs when compared to 0% DOTAP, and a 10-fold increase when compared to 100% DOTAP at the 4-hour time point. Notably, a significant increase was also observed at the 24-hour incubation time point (FIG. 9C).

In addition to in vitro data, the role of DOTAP in the LNP formulation in the context of delivery of an mRNA encoding a reporter gene to SMCs in vivo was confirmed. In this example, the effect of DOTAP as a fifth component added to LNPs and its ability to deliver mRNA to SMCs in vivo in a Marfan disease mouse model was studied. Using DOTAP particles with 10%, 50%, and 80% concentration, mRNA expressing a Cre recombinase enzyme was delivered. The Cre recombinase enzyme enables the expression of tdTom protein in this genetically engineered model. It was found that when comparing the LNPs of the art to the instant LNPs comprising DOTAP, the same extent of tdTom expression is achieved in certain organs.

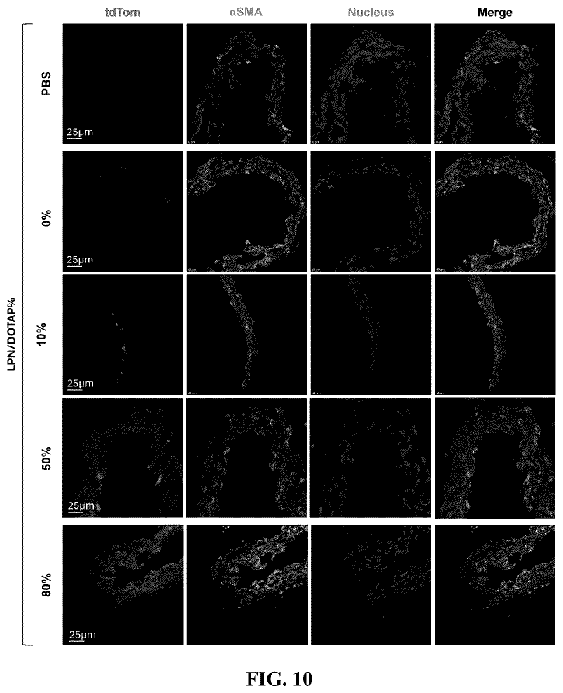

Increasing concentrations of DOTAP were also evaluated. FIG. 10 are histological images indicating that increasing the % of DOTAP in LNP formulation increases SMC tdTom expression In vivo. In the experiment results shown in FIG. 10, Cre-mRNA delivery utilizing LNPs with tested with increasing DOTAP content and expression of tdTom in a Marfan disease mouse model. Mice at P3 were injected with 1 mg/kg mRNA encapsulated in LNPs formulated with 0, 10, 50 and 80% DOTAP lipid. tdTom expression identified using immune fluorescence in histological sections of the aorta and localized to SMCs (indicated by α-SMA expression).

Thus, it was demonstrated that DOTAP (7% to 80%) comprising LNPs display preferential delivery towards smooth muscle cells in vivo, as compared to standard LNPs, in different mouse models and with two different cargos (plasmid and mRNA).

### Example 5: Targeting LNPs to Mutant Smooth Muscle Cells Using Targeting Peptides

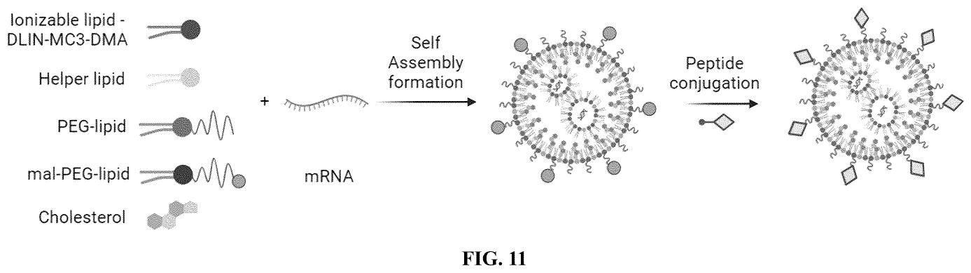

In order to enhance the targeting, and potentially uptake, of the nanoparticles to specific tissue or cells, DOTAP LNPs conjugated with peptides that can target smooth muscle cell tissue or cell surface receptors were developed. The targeting strategy was composed of two approaches. First, conjugating our DOTAP nanoparticles with peptides that target receptors highly expressed in diseased vasculature extracellular matrix (collagen IV) in order to increase the accumulation and the retention of the nanoparticles in diseased tissues. Second, conjugating our DOTAP nanoparticles with peptides that target receptors highly expressed on the surface of vSMCs (IL-6R, CD63, and GAL-3) increasing the uptake into these cells. A combination of the two approaches can potentially leverage both accumulation and retention at the target site and cell specificity. FIG. 11 is a schematic illustrating the aforementioned conjugation scheme. LNPs were conjugated with the desired peptide by swapping traditional PEG lipids typically present in standard LNP formulations with maleimide-terminally modified PEG lipid. Peptide conjugation was done in different densities controlled by the percentage of the maleimide-terminally modified PEG lipid in the LNP formulation, ranging from 0.15-1.2% (FIG. 11). Peptide sequences targeting extracellular matrix and cellular receptors are described in Table 4 below:

TABLE 4
Peptide sequences targeting extracellular
matrix and cellular receptors.

| Peptide receptor | Sequence |
|---|---|
| Col-4 | KLWVLPK-GGG-C; SEQ ID NO: 22 |
| IL6-R | C-GGG-LSLITRL; SEQ ID NO: 23 |
| CD63 | CRHSQMTVTSRL; SEQ ID NO: 24 |
| Gal-3 | C-GGG-ANTPCGPYTHDCPVKR; |
| | SEQ ID NO: 25 |

To evaluate this approach, LNPs conjugated with different peptides were first synthesized, their physical properties were characterized, and their functional activity in MOVAS cell line in vitro was examined.

Table 5 summarizes the physical properties of the assembled LNPs conjugated or non-conjugated with the listed peptides in changing surface densities controlled by the percentage of a linker lipid in the LNP formulation. Increasing linker percentages (0.15-1.2%) increases the conjugated peptides on LNPs surface which confirms the chemical conjugation to the surface of the LNPs. The physical properties highly affect the potential functionality of the LNPs therefore crucial to characterize. The physical properties of the LNPs post conjugation present a slight increase in size with no significant effect on PDI, however, not affected by the percent of conjugated peptide. On the other hand, zeta potential decreases while increasing the percentage of the conjugated peptide.

TABLE 5
Physical properties (size, PDI, and Zeta potential) of
peptide-conjugated and non-conjugated LNPs by Col4/IL6R/CD63/Gal3
targeting peptides in varying densities.

| | Size (nm) | PDI | Zeta potential (mV) |
|---|---|---|---|
| LNP-DOTAP | 131.8 | 0.14 | −5.48 |
| 0.15% NC | 145.2 | 0.15 | −5.47 |
| 0.3% NC | 161.1 | 0.24 | −4.24 |
| 0.6% NC | 137.8 | 0.17 | −4.17 |
| 0.9% NC | 136.3 | 0.19 | −4.39 |
| 1.2% NC | 118.6 | 0.19 | −5.89 |
| 0.15% Col-4 | 188.33 | 0.17 | −6.79 |
| 0.3% Col-4 | 144.20 | 0.26 | −7.00 |
| 0.6% Col-4 | 165.63 | 0.14 | −11.87 |
| 0.9% Col-4 | 148.83 | 0.19 | −14.33 |
| 1.2% Col-4 | 139.43 | 0.15 | −17.30 |
| 0.15% IL-6R | 131.97 | 0.25 | −7.66 |
| 0.3% IL-6R | 111.51 | 0.33 | −7.45 |
| 0.6% IL-6R | 60.24 | 0.57 | −9.78 |
| 0.9% IL-6R | 137.43 | 0.19 | −11.70 |
| 1.2% IL-6R | 140.30 | 0.07 | −13.90 |
| 0.15% CD-63 | 125.2 | 0.104 | 1.36 |
| 0.3% CD-63 | 120.2 | 0.047 | −3.67 |
| 0.6% CD-63 | 118.3 | 0.06 | −10.4 |
| 0.9% CD-63 | 195.1 | 0.24 | −13.5 |
| 1.2% CD-63 | 126.5 | 0.088 | −12.3 |
| 0.15% Gal-3 | 134.1 | 0.094 | −0.975 |
| 0.3% Gal-3 | 131.9 | 0.089 | −3.74 |
| 0.6% Gal-3 | 120.4 | 0.102 | −5.9 |
| 0.9% Gal-3 | 119.9 | 0.058 | −10.4 |
| 1.2% Gal-3 | 113 | 0.128 | −13.4 |

LNPs Decorated with Collagen IV (Col-IV) Peptides

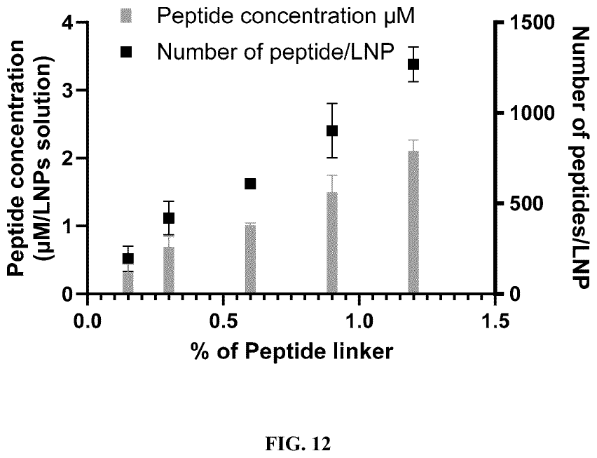

In addition, based on nanoparticle tracking analysis, the percentage of linker lipid was estimated and correlated to the concentration of conjugated peptide. Furthermore, the number of peptide units per LNP was plotted (FIG. 12) resulting in a range varying from about 194 peptides to about 1268 per LNP indicating the density of peptides per particle.

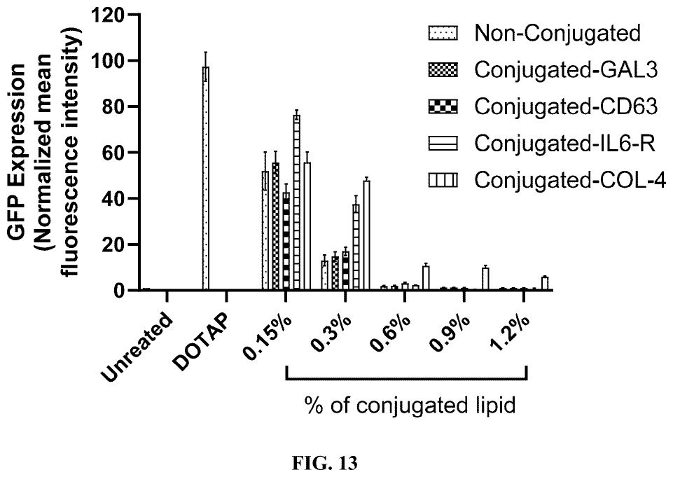

Following the synthesis and characterization, the efficiency of conjugated LNPs was first examined in functional assays in vitro. MOVAS cells were treated with peptide-conjugated LNPs encapsulating mRNA expressing GFP. GFP expression was analyzed by flow cytometry 24 h post treatment. The results in FIG. 13 demonstrate the extent of GFP expression as a function of conjugated peptide content and sequence. The efficiency of mRNA expression was highly affected by the peptide amount, demonstrating an optimum at 0.15-0.3% of conjugated peptide for in vitro application regardless of the peptide sequence. The addition of the lipid with the linker, even without the peptides, was also affected by the linker lipid content (non-conjugated), indicating that LNPs composed of high percentages of lipid linker affected their functional activity. It was then confirmed that a range for peptide conjugation was identified, which remained unaffected by the additional manipulations used for peptide conjugation, despite the impact of high percentages of lipid linker on the functional activity of LNPs.

It was therefore optimized and confirmed that peptide-conjugated LNPs efficiently expressed GFP in vitro, suggesting that the conjugation did not moderate their functional activity, thereby encouraging the examination of the potential of targeting SMCs in vivo.

The incorporation of DSPE-PEG-maleimide (DSPE-PEG-mal) lipid into the formulation is critical, as it plays a key role in stabilizing the outermost shell of the lipid nanoparticle (LNP) and maintaining the presentation of the conjugated peptide. However, while increasing the proportion of this moiety enhances stability, excessive amounts may lead to reduced particle uptake and hinder disassembly resulting in over-stabilization. Therefore, it is essential to identify an optimal concentration, as demonstrated in our study.

### Example 6: Targeting Peptide-Conjugated Lipid Nanoparticles for Enhanced Delivery to Vascular Smooth Muscle Cells

In some aspects, the present invention relates to lipid nanoparticles (LNPs) conjugated with targeting peptides for enhanced delivery to vascular smooth muscle cells (vSMCs). Following the in vitro validation of functional activity, we evaluated the ability of mRNA-loaded LNPs to selectively target VSMCs in vivo. The formulations shown in Table 6 were tested.

TABLE 6
LNP formulations.

| Formulation | MC-3 | DOPE | Cholesterol | DMG-PEG | DSPE-PEG-mal | DOTAP |
|---|---|---|---|---|---|---|
| 0.15% NC | 0.450 | 0.0945 | 0.3420 | 0.0120 | 0.0015 | 0.100 |
| 0.3%-Col-IV | 0.450 | 0.0945 | 0.3420 | 0.0105 | 0.0030 | 0.100 |
| 0.3% NC | 0.450 | 0.0945 | 0.3420 | 0.0105 | 0.0030 | 0.100 |
| 0.6%-Col-IV | 0.450 | 0.0945 | 0.3420 | 0.0075 | 0.0060 | 0.100 |
| 0.6% NC | 0.450 | 0.0945 | 0.3420 | 0.0075 | 0.0060 | 0.100 |
| LNP/DOTAP/80% | 0.100 | 0.021 | 0.076 | 0.003 | 0.000 | 0.800 |
| 0.3%-Col-IV | 0.100 | 0.0210 | 0.0760 | 0.0120 | 0.0030 | 0.7880 |
| (80% DOTAP) | | | | | | |
| 0.6%-Col-IV | 0.100 | 0.0210 | 0.0760 | 0.0090 | 0.0060 | 0.7880 |
| (80% DOTAP) | | | | | | |
| 0.3%-IL-6R | 0.450 | 0.0945 | 0.3420 | 0.0105 | 0.0030 | 0.100 |
| 0.15%-Col-IV + | 0.450 | 0.0945 | 0.3420 | 0.0105 | 0.0030 | 0.100 |
| 0.15%-IL-6R | | | | | | |

The LNPs are designed to encapsulate nucleic acids and are conjugated with peptides targeting extracellular matrix components or cell surface receptors. This technology demonstrates improved targeting and gene expression in vSMCs in mouse models of vascular diseases, in part by targeting receptors in vSMCs.

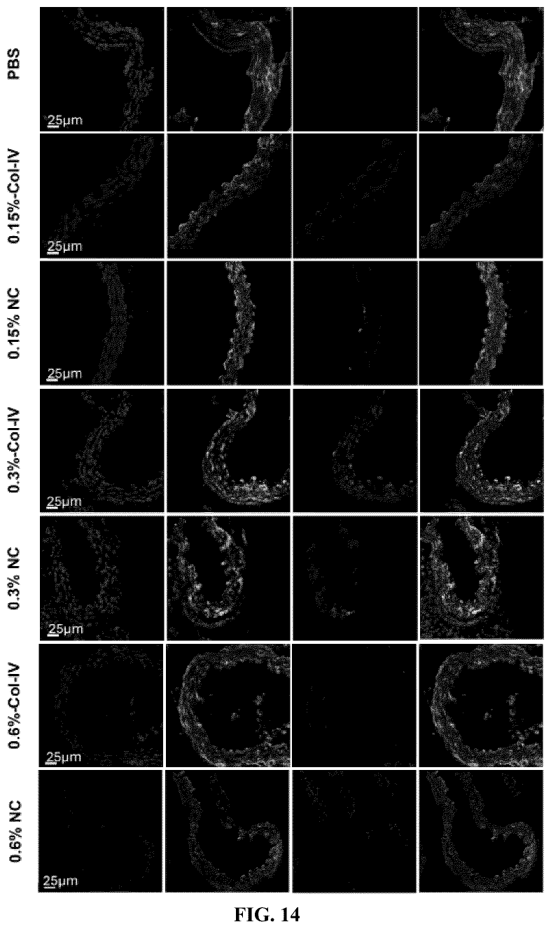

To achieve this, we encapsulated Cre-mRNA in LNPs, which were subsequently conjugated with a Collagen IV-targeting peptide, leveraging its affinity for the extracellular matrix of VSMCs. We hypothesized that Col-IV peptide conjugation would enhance LNP retention within the VSMC extracellular matrix, thereby increasing bioavailability and uptake by SMCs. To test this, Marfan mice were injected at P3 with Col-IV-conjugated LNPs, and tdTom fluorescence expression was analyzed six days post-injection. Additionally, we sought to determine the optimal peptide density on the LNP surface for maximal retention and subsequent SMC uptake, leading to tdTom expression (Table 6). We evaluated three peptide densities, 0.15%, 0.3%, and 0.6% relative to the lipid component PEG-DSPE, into which the peptide was conjugated. FIG. 14 presents histological images of mice aorta, revealing that LNPs conjugated with Col-IV at 0.3% peptide density exhibited the most efficient SMC-specific tdTom expression. Moreover, this enhanced expression was significantly higher compared to non-conjugated LNPs, reinforcing the effectiveness of Col-IV peptide-mediated targeting.

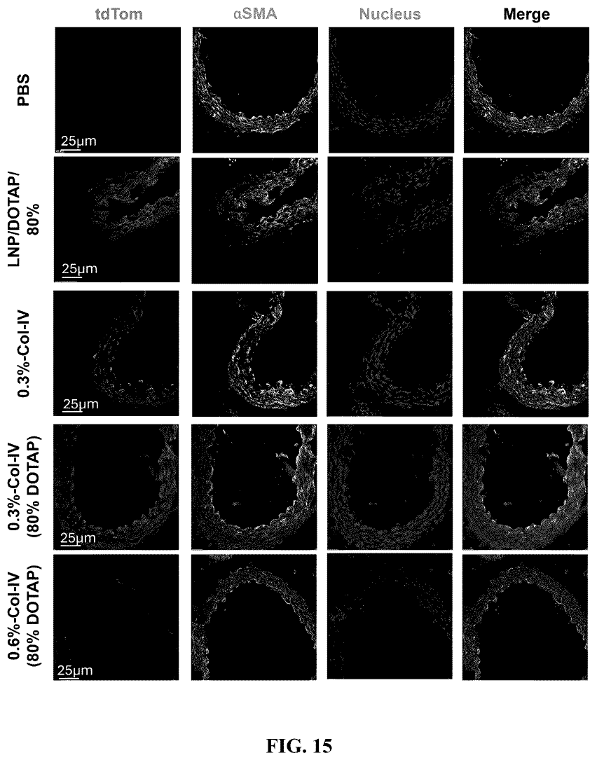

In addition, we have explored for potential synergy between the two examined elements, peptide-based targeting and the addition of DOTAP lipid to the formulation, in increasing the targeting efficiency for SMCs. We have formulated Cre-mRNA in a formulation containing 80% DOTAP lipid which was then conjugated by Col-IV targeting peptide and tdTom expression was examined 6 days post injection. FIG. 15 presents histology sections of the aorta demonstrating that there was no increase in tdTom SMC expression for the combined Col-IV-DOTAP approach in comparison to the single approach where we either use the optimal formulation (80% DOTAP) to target SMCs or decorate the LNPs with Col-IV targeting peptide. As shown in FIG. 15, leveraging two approaches to target SMCs doesn't demonstrate an increase in SMC tdTom expression. LNPs composed of the 80% DOTAP formulation were conjugated by Col-IV peptide and compared to each of the counterparts' controls that were found most effective as a single approach (80% DOTAP formulation or 0.3%-Col-IV conjugated LNP). In addition, the effect of % of Col-IV peptide decoration on the 80% DOTAP formulation was examined to verify optimal combination. Cre-mRNA encapsulated in each of the LNPs was injected into Marfan disease mouse models at P3 at a dose of 1 mg/kg mRNA. Six days post-injection, tdTom expression was assessed via immunofluorescence in histological sections of the aorta, revealing localization within smooth muscle cells (SMCs), identified by α-SMA staining.

### Example 7: Targeting Inflammation Using Peptide-Conjugated LNPs

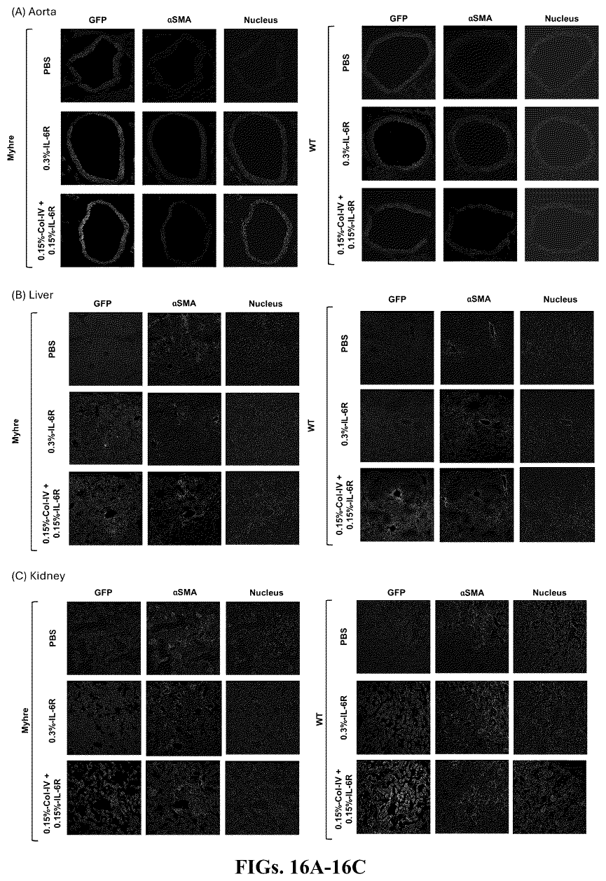

To explore the potential of targeting inflamed tissues in adult animals we have conjugated LNPs with peptide targeting IL-6R which is overexpressed in situations where inflammation is involved. In addition, we have designed an LNP formulation where the two peptides were conjugated to the same LNP as a means to enhance tissue targeting and cellular targeting at the same delivery strategy. Therefore, IL-6R targeting peptide was conjugated in combination with Col-IV targeting peptide. To test our hypothesis, we encapsulated mRNA expressing GFP and examined expression using histology imaging and analysis and injected three month old (adult) wild type mice and mice engineered with a gain of function mutation in SMAD4 to model the human condition, Myhre syndrome both fed a high fat diet. We found increased GFP expression in examined organs (aorta, kidney, liver) with the LNPs that were conjugated with both peptides in comparison to a single IL-6R peptide conjugation (FIGS. 16A-C). It is important to note that even the wildtype mice have elevated inflammation due to the high fat diet the mice are fed with causing this difference to appear in some cases mainly in the liver and kidney, but not the aorta.

We verified the results obtained in the Myhre syndrome mouse model in an LDLR knockout mouse model which is used as a model for inflamed vascular disease indications. GFP mRNA was encapsulated in LNPs conjugated by IL-6R targeting peptide (0.3%). 12 h post intravenous injection we examined GFP expression in the aorta using histology analysis. While comparing conjugated to non-conjugated formulation, we identify a significant increase in GFP expression using the IL-6R targeting peptide formulation showing that the peptide sequence on the surface of the LNPs is crucial in order to increase the encapsulated mRNA in smooth muscle cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A particle comprising:
a) an amount of an ionizable lipid;
an amount of neutral lipid;
an amount of cholesterol; and
an amount of one or more PEG-lipids;
an amount of a DOTAP molecule; and
b) a peptide conjugated to a linker in the particle.

2. The particle of claim 1, wherein the linker is a maleimide group at a PEG lipid of the one or more PEG-lipids in the particle.

3. The particle of claim 2, wherein the linker is a maleimide-terminally modified PEG lipid.

4. The particle of claim 1, wherein the one or more PEG-lipids comprise 1,2-dimyristoyl-rac-glycero-3-methoxypolyethylene glycol (DMG-PEG) and 1,2-distearoyl-sn-glycero-3-phosphoethanolamine-N-[maleimide(polyethylene glycol](DSPE-PEG-maleimide).

5. The particle of claim 1, wherein the peptide is a peptide targeting collagen IV (Col-IV) or a functional fragment thereof.

6. The particle of claim 1, wherein the peptide is a peptide targeting IL-6R.

7. The particle of claim 1, wherein the peptide is a peptide targeting CD63.

8. The particle of claim 1, wherein the peptide is a peptide targeting GAL-3.

9. The particle of claim 1, further comprising:
amounts of the ionizable lipid, the neutral lipid, the cholesterol, the one or more PEG-lipids, and DOTAP at a molar ratio of 10:2.1:7.6:1.5:78.8.

10. The particle of claim 1, wherein the neutral lipid is a phosphatidylcholine lipid or a phosphatidylethanolamine lipid.

11. The particle of claim 10, wherein the phosphatidylcholine lipid or the phosphatidylethanolamine lipid is selected from the group consisting of DOPE, DOPC, DSPC, DPPC, POPC, and SOPC.

12. The particle of claim 1, wherein the ionizable lipid is selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DM A, DLin-K-C4-DMA, DLen-C2K-DMA, 7-DLen-C2K-DMA, or DLin-MP-DMA.

13. The particle of claim 1, wherein the particle encapsulates a nucleic acid therapeutic cargo.

14. The particle of claim 13, wherein the therapeutic cargo is an mRNA molecule encoding a gene, optionally a gene for rescuing gene expression in a smooth muscle cell.

15. The particle of claim 13, wherein the therapeutic cargo is a plasmid encoding a gene, optionally a gene for rescuing gene expression in a smooth muscle cell.

16. The particle of claim 15, wherein the therapeutic cargo comprises a nucleic acid molecule encoding an ectonucleotide pyrophosphatase/phosphodiesterase 1 (ENPP1) therapeutic cargo, a nucleic acid molecule encoding an ATP Binding Cassette Subfamily C Member 6 (ABCC6) therapeutic cargo, a nucleic acid molecule encoding an Actin Alpha 2 (ACTA2) gene, or a nucleic acid encoding a genome editing protein.

17. The particle of claim 16, wherein the ENPP1 therapeutic cargo encodes a transmembrane ENPP1 molecule.

18. The particle of claim 17, wherein the transmembrane ENPP1 molecule is SEQ ID NO: 1.

19. The particle of claim 16, wherein the ENPP1 therapeutic cargo encodes a soluble ENPP1 molecule.

20. The particle of claim 19, wherein the soluble ENPP1 molecule comprises amino acids 103-925, and optionally amino acids 97-925 of SEQ ID NO: 1.
